(19) United States
(12) Patent Application Publication
(10) Pub. No.: US 2003/0180756 A1
(43) Pub. Date: Sep. 25, 2003

(54) Compositions and methods for suppressing eukaryotic gene expression

(76) Inventors: Yang Shi, Brookline, MA (US); Guangchao Sui, Malden, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/301,516

(22) Filed: Nov. 21, 2002

Related U.S. Application Data
(60) Provisional application No. 60/366,478, filed on Mar. 21, 2002.

(51) Int. Cl. C12Q 1/68; C07H 21/04; C12N 9/22; A61K 48/00; C12P 21/02; C12N 5/06

(52) U.S. Cl. 435/6; 435/69.1; 435/199; 435/320.1; 435/325; 514/44; 536/23.2

(57) ABSTRACT

The invention provides compositions and methods for suppressing gene expression in cells, in particular in eukaryotic cells. Nucleic acids encoding RNAs targeting specific genes and thereby inhibiting expression of these genes are provided. The RNAs may form hairpin structures. The nucleic acids may be included in a vector. The compositions can be used for treating various diseases by inhibiting the expression of abnormal, e.g., mutated proteins.

### CROSS-REFERENCE TO RELATED APPLICATIONS

This application claims the benefit of U.S. Provisional Application No. 60/366,478, filed Mar. 21, 2002, the contents of which are specifically incorporated by reference herein.

### GOVERNMENT SUPPORT

This invention was made with government support under grant No. GM53874 awarded by the National Institutes of Health. The government has certain rights in the invention.

### BACKGROUND OF THE INVENTION

Numerous diseases, in particular malignant diseases, are associated with expression of a mutant protein. For example, leukemias are often associated with chromosomal translocations resulting in expression of fusion genes that can be causally implicated in disease pathogenesis. Until recently, these diseases have been treated with empirically derived cytotoxic chemotherapy that is marginally more toxic for leukemic cells than normal hematopoietic progenitors. For example, most adults who develop acute leukemia will die of their disease of complications of therapy. Thus, there is a clear need to develop more effective and less toxic therapies for leukemia.

Double-stranded RNA (dsRNA) can trigger silencing of homologous gene expression by a mechanism termed RNAi (for RNA-mediated interference) (1). RNAi is an evolutionarily conserved phenomenon and a multistep process that involves generation of active small interfering RNA (siRNA) in vivo through the action of an RNase III endonuclease, Dicer. The resulting 21- to 23-nucleotides siRNA mediates degradation of the complementary homologous RNA (reviewed in refs. 2-4). RNAi has been used as a reverse genetic tool to study gene function in multiple model organisms, including plants, Caenorhabditis elegans, and Drosophila where large dsRNAs efficiently induce gene-specific silencing (1, 5-7).

One obstacle to achieving RNAi in mammals is that dsRNAs longer than 30 nucleotides will activate an antiviral response, leading to the nonspecific degradation of RNA transcripts and a general shutdown of host cell protein translation (8, 9). As a result, the long dsRNA, with a few exceptions (10, 11), does not produce RNAi activity, and RNAi therefore is not a general method for silencing specific genes in mammalian cells. This obstacle has been recently overcome by Tuschl and colleagues (12) who found that gene-specific suppression in mammalian cells can be achieved by vitro-synthesized siRNA that are 21 nucleotides in length, long enough to induce gene-specific suppression, but short enough to evade the host interferon response. However, it would be difficult to introduce sufficient siRNA into cells, e.g., for treating diseases such as leukemias. Accordingly, a method for producing siRNA in cells in sufficient quantity for inhibiting a target gene, is highly desirable.

### SUMMARY OF THE INVENTION

In one embodiment, the invention provides nucleic acids comprising the following nucleotide sequences in a 5′ to 3′ order: an RNA polymerase promoter sequence; a first target sequence that is essentially complementary to a sequence of a target nucleic acid or complement thereof; a spacer sequence; a second target sequence that is essentially complementary to the first target sequence; and an RNA polymerase termination signal, wherein an RNA transcribed from the nucleic acid can inhibit expression of the target gene. The RNA transcribed from the nucleic acid may form a hairpin structure, wherein the first and the second target sequences essentially form the stem of the hairpin and the spacer essentially forms the loop at the closed end of the hairpin. The RNA may be an siRNA. The polymerase may be an RNA polymerase III (Pol III), e.g., a U6 promoter, which may comprise at least part of about nucleotide −315 to about nucleotide +1 of the mouse U6 promoter (SEQ ID NO: 3). The polymerase termination signal may comprise a number of thymidines sufficient for arresting Pol III activity, 2, 3, 4 or 5 thymidines.

The first target sequence may be at least about 95% identical, and is preferably perfectly complementary, to a nucleotide sequence of the target nucleic acid or the complement thereof. The target nucleic acid may be a target gene, e.g., a gene in the genome of a cell. The first and the second target sequences may comprise from about 15 to about 30 nucleotides; from about 19 to 25 nucleotides; about 20 or about 21 nucleotides of a target nucleic acid or complement thereof. The first target sequence may comprise a portion of the coding sequence of the target nucleic acid or the complement thereof. The first and the second target sequences may differ in at most 2 nucleotides or they may be perfectly complementary.

The spacer sequence may consist of about 3 to about 15 nucleotides; about 5 to about 10 nucleotides; or about 6 nucleotides. The spacer sequence may comprise or consist of a palindromic sequence, e.g., that of a restriction enzyme recognition site, such as AACGTT.

The nucleic acid may be DNA. It may be in a plasmid. Or in an expression vector, e.g., a eukaryotic expression vector, which may be a mammalian expression vector. The eukaryotic expression vector may a viral vector, e.g., an adenoviral or AAV vector.

In a preferred embodiment of the invention, the polymerase is a Pol III; the first target sequence is essentially complementary to a sequence of a target nucleic acid or complement thereof; the first and the second target sequences consist of about 19-23 nucleotides and are perfectly complementary to each other; the spacer sequence consists of about 6 nucleotides; and the RNA polymerase termination signal consists of 4 or 5 thymidines.

In another embodiment, the invention provides nucleic acids comprising the following nucleotide sequences in a 5′ to 3′ order: a polymerase, e.g., Pol III, promoter sequence; a first restriction enzyme recognition sequence; a spacer sequence; a second restriction enzyme recognition sequence; and a polymerase termination signal, e.g., a number of thymidines sufficient for arresting Pol III activity, wherein an RNA molecule transcribed from the nucleic acid in which a first and a second target sequences are inserted in the first and second restriction enzyme recognition site, respectively, inhibits expression of a target gene comprising a sequence that is essentially complementary to the first or the second target sequence. The spacer sequence; polymerase promoter; and polymerase termination signal may be as described above. The nucleic acid may further comprise at least one additional restriction enzyme recognition sequence located, e.g., between the Pol III promoter and the first restriction enzyme recognition sequence. It may also further comprise at least one additional restriction enzyme recognition sequence located, e.g., between the second restriction enzyme recognition sequence and the thymidines sufficient for arresting Pol III activity.

Also within the scope of the invention are RNAs comprising the following nucleotide sequences in a 5′ to 3′ order: a first target sequence of about 19 to about 25 nucleotides, which is essentially complementary, e.g., at least about 95% identical, to a portion of a nucleotide sequence of a target nucleic acid or the complement thereof; a spacer sequence of about 5 to 10 nucleotides; a second target sequence of about 19 to about 25 nucleotides that is essentially complementary to the first target sequence; and at least a portion of an RNA polymerase termination signal, wherein the RNA inhibits expression of a target gene comprising a sequence that is essentially complementary to the first or the second target sequence. The RNA may forms a hairpin structure. It may have the structure of an siRNA. The first and the second target sequences may consist of about 19 to about 23 nucleotides and may be perfectly complementary to each other. The first target sequence may be perfectly complementary to a sequence of the target nucleic acid or complement thereof. The polymerase termination signal may consist of 2, 3, 4 or 5 uridines.

Also provided are cells comprising a nucleic acid described herein. The cell may be a eukaryotic cell, such as a mammalian cell. The cell may be an isolated cell, it may be in vitro or in vivo.

In another embodiment, the invention provides methods for preparing a nucleic acid for inhibiting the synthesis of a target protein in a eukaryotic cell, comprising (i) providing a nucleic acid described herein; and (ii) introducing into the first restriction recognition sequence a first oligonucleotide of about 15-30 nucleotides comprising a sequence that is essentially complementary to a sequence of the target nucleic acid. The method may further comprise introducing into the second restriction recognition sequence a second oligonucleotide of about 15-30 nucleotides that is essentially complementary to the sequence of the first oligonucleotide. The first oligonucleotide may comprise about 19 to 23 consecutive nucleotides of the target nucleic acid or the complement thereof. The second oligonucleotide may comprise a nucleotide sequence that is perfectly complimentary to the sequence of the first oligonucleotide.

Also provided are methods for producing RNA molecules that inhibit expression of a target nucleic acid in eukaryotic cells, comprising introducing into a eukaryotic cell a nucleic acid described herein, wherein the first target sequence is essentially complementary to a sequence of the target nucleic acid or the complement thereof, such that the nucleic acid is transcribed in the eukaryotic cell and produces RNA molecules that inhibit expression of a target nucleic acid.

In yet another embodiment, the invention provides methods for inhibiting the synthesis of a target protein in a eukaryotic cell, comprising, e.g., introducing into a target cell a nucleic acid described herein, wherein the first target sequence is essentially complementary to a sequence of the nucleic acid encoding the target protein or the complement thereof, such that the nucleic acid is transcribed in the target cell and thereby inhibits the synthesis of the target protein. The cell may be an isolated cell or a cell in an organism, such as a subject. The method may comprise first obtaining the cell from a subject; introducing the nucleic acid into the cell ex vivo and administering the cell to the subject.

Also provided are kits for inhibiting the synthesis of a target protein in a cell, comprising, e.g., a nucleic acid described herein and optionally at least one reagent for introducing the nucleic acid into a cell.

### BRIEF DESCRIPTION OF THE FIGURES

FIG. 1A is a diagram showing a construct for generating siRNA from DNA template including: a mouse U6 promoter; a 21 nucleotide long sequence corresponding to a portion of the coding region of the gene of interest inserted at the +1 position of the U6 promoter (−315 to +1); a spacer of 6 nucleotides; a 21 nucleotide long sequence that is the complementary sequence to the other 21 nucleotide sequence in the construct; and a transcriptional termination signal of five thymidines at the 3′ end of the inverted repeat, and the resulting RNA that is predicted to fold back to form a hairpin double stranded (ds) RNA.

FIG. 1B shows the fluorescence, provided by the presence of GFP, in HeLa cells cotransfected with BS/U6 (i.e., empty vector), CMV-GFP and CMV-HA-ERK-5 plasmids (a-c) or with BS/U6/gfp, CMV-GFP and CMV-HA-ERK-5 plasmids (d-f),showing the presence of GFP (a and d); the presence of HA-ERK-5 (b and e) and all the cells in the field, as detected by staining with 4′,6-diamidino-2-phenylindole (DAPI). Solid arrows indicate cells that are positive for HA-ERK-5 but display nearly undetectable GFP levels. All corresponding images were taken at the same exposure and a magnification of ×60.

FIG. 1C depicts a Western blot showing the level of GFP and HA-ERK-5 in cells cotransfected with either BS/U6 (first colomn) or 1.5 or 3.0 μg BS/U6/gfp (second and third column, respectively) and CMV-HA-ERK-5.

FIG. 2 shows HeLa cells transfected with either the BS/U6 vector (a-c and j-l) or the RNAi plasmid BS/U6/lamin A/C (last two columns) together with CMV-GFP, and stained with the anti-lamin A/C antibody (a and d); with secondary antibody only (g and p); showing the fluorescence caused by the presence of GFP (b, e, h, k, n, and q); stained with antibodies that recognized the related lamin B protein (j and m); or stained with 4′,6-diamidino-2-phenylindole (DAPI), showing all cells (c, f, i, l, o and r) a, b and c; d, e and f; g, h and i; j, k and l; m, n and o; p, q and r show the same sets of cells, respectively. Corresponding images were taken at the same exposure and at a magnification of ×60.

FIG. 3A shows HeLa cells transfected with either the BS/U6 vector (a-c) or the BS/U6/cdk-2 vector (d-f and g-I) and CMV-GFP, and stained with anti-CDK2 antibody (a and d); stained with secondary antibody only (g); showing the fluorescence caused by the presence of GFP (b, e and h); or stained with 4′,6-diamidino-2-phenylindole (DAPI), showing all cells (c, f and i). Solid arrows indicate two of the GFP-positive cells (transfected cells) in which CDK-2 expression was below the level of detection. Open arrows indicate two GFP-negative cells in which CDK-2 expression was also undetectable.

FIG. 3B shows HeLa transfected with either BS/U6 vector (a-c) or BS/U6/dnmt-1 (d-f and g-i) and CMV-GFP, and stained with the anti-DNMT-1 antibody (a and d); with secondary antibody only (g); showing the fluorescence caused by the presence of GFP (b, e, and h); or stained with 4′,6-diamidino-2-phenylindole (DAPI), showing all cells (c, f, and i,). Solid arrows indicate two GFP-positive cells (transfected cells) in which DNMT-1 expression was barely detectable. Open arrows indicate two GFP-negative cells in which DNMT-1 expression is also undetectable.

FIG. 4A shows the structure of an AAV2 vector encoding an RNA inhibiting gene expression and a GFP protein.

FIG. 4B depicts a protein blot showing YY1, CDK2 and CtBP1 protein levels in cells incubated with an AAV2 vector encoding an RNA targeting YY1 (“YY1:Ri”), CDK2 (“CDK2:Ri”) or an empty vector (“Contr.”).

FIG. 5A shows the nucleotide sequence of p11 (SEQ ID NO: 24), p10 (SEQ ID NO: 25) and p9 (SEQ ID NO: 26) siRNAs targeting the G256C mutant SOD1 gene; the nucleotide sequences of the the wild-type (SEQ ID NO: 27) and mutant G256C (SEQ ID NO: 28) genes encompassing the mutation; and the p9 (SEQ ID NO: 29), p10 (SEQ ID NO: 30) and p11 (SEQ ID NO: 31) siRNAs targeting the wild-type construct.

FIG. 5B shows in vitro RNAi experiments targeting mutant or wild-type SOD1 mRNA with mutant or wild-type siRNAs.

FIG. 5C shows the fraction of mRNA remaining or the amount of cleavage product produced as a function of time, showing that mutant siRNA p10 targets mutant but not wild-type SOD1 mRNA for destruction by the RNAi pathway.

FIG. 6 shows the relative number of green and red HeLa cells, as determined by FACS, transfected with SOD1wtGFP or SOD1-G85R-GFP and siRNA p9, p10 or p11 of SOD1 wild-type (wt) or SOD1 G85R.

FIG. 7A shows the nucleotide sequence of wild-type SOD1 encompassing the nucleotide at position 281 that can be mutated (SEQ ID NO: 32), and the nucleotide sequence of the siRNA targeted at the G281C mutant of this region (G93A shRNA) (SEQ ID NO: 33).

FIG. 7B shows the relative number of green and red cells, as determined by FACS, transfected with SOD1wtGFP or G93A-GFP and U6-empty vector or U6-G93A.

FIG. 8A shows the relative number of green and red neuroblastoma N2a cells, as determined by FACS, transfected with SOD1wtGFP or SOD1-G85R-GFP and siRNA targeting luciferase or with siRNA p10SOD1wt or p10SOD1G85R.

FIG. 8B shows the relative number of green and red neuroblastoma N2a cells, as determined by FACS, transfected with SOD1wtGFP or SOD1-G93A-GFP and U6 empty vector, U6-G93A vector or U-6G93A and SOD1-GFP vectors at different ratios.

FIG. 9A shows protein blots of transfected HeLa cells detecting G85R-GFP or endogenous human SOD1, wherein the HeLa cells were transfected with nothing (“no siRNA”), or with 20 or 2 nM of siRNA targeting luciferase; siRNA targeting wild-type SOD1 (“SOD1wt”); siRNA targeting SOD1G85R (“SOD1G85R”); or with 3′blocked siRNA targeting SOD1G85R (last lane).

FIG. 9B shows the relative levels of SOD1 measured from the protein blots of FIG. 9A.

FIG. 10A shows a protein blot of liver proteins from mice to which SOD1-G93A-GFP and C-terminal myc tagged wild-type human SOD1 were administered, showing SOD1 G93A-GFP protein, SOD1 wt-myc protein or mouse SOD1.

FIG. 10B shows the relative intensities of the proteins in FIG. 10A.

### DETAILED DESCRIPTION OF THE INVENTION

Definitions

For convenience, the meaning of certain terms and phrases employed in the specification, examples, and appended claims are provided below. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise.

When referring to a sequence that “consists of about” a certain number of nucleotides, this is intended to refer to a sequence that consists of the certain number of nucleotides plus or minus 20% or 10% of the number of nucleotides. For example, a sequence consisting of about 10 nucleotides refers to a sequence of 8, 9, 10, 11 or 12 nucleotides.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of a nucleic acid of interest due to the degeneracy of the genetic code.

“Essentially complementary” when referring to two nucleic acid strands refers to nucleic acid strands that are sufficiently complementary to allow hybridization of the two strands under the desired conditions. Accordingly, the two strands may be at least 90%, preferably at least 95% or 98% complementary. In other words, the two strands may differ in at most 5, 4, 3, 2 or 1 nucleotides.

A “hairpin structure” when referring to the structure of a nucleic acid, refers to a single stranded nucleic acid in which two portions of the nucleic acid hybridize to each other to form the stem of a hairpin structure and a sequence located between the two portions forms a loop at one end of the stem.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes that contain certain mismatches, provided that the two strands are still forming a double stranded helix. “Stringent hybridization conditions” refers to hybridization conditions resulting in essentially specific hybridization.

“Inhibiting gene expression” refers to any action that results in decreased production of a polypeptide encoded by the gene or decreased levels of an RNA encoded by the gene. Inhibiting gene expression includes inhibiting transcription, translation or degrading the DNA template or RNA encoded thereby.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding a polypeptide preferably includes no more than 10 kilobases (kb) of nucleic acid sequence which naturally immediately flanks the gene encoding the polypeptide in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A recombinant protein is a non-naturally-occurring protein.

“Non-human animals” of the invention include mammalians such as rodents, non-human primates, sheep, dog, cow, chickens, amphibians, reptiles, ovines, bovines, equines, canines, felines etc.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrase “nucleic acid corresponding to a gene” refers to a nucleic acid that can be used for detecting the gene, e.g., a nucleic acid which is capable of hybridizing specifically to the gene.

A nucleic acid is “operably linked” to another nucleic acid when it is placed into a functional relationship with another nucleic acid sequence. For example, a promoter or enhancer is operably linked to a coding sequence if it affects the transcription of the sequence. Generally, “operably linked” means that the DNA sequences being linked are contiguous. However, they can also be separated by other DNA sequences. Linking is accomplished by ligation at convenient restriction sites. If such sites do not exist, the synthetic oligonucleotide adaptors or linkers are used in accordance with conventional practice.

“Percent identity” between two amino acid sequences or between two nucleotide sequences can be determined by comparing a position in each sequence that may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

A nucleotide sequence is “perfectly complementary” or “perfectly matched” to another nucleotide sequence if each of the bases of the two sequences match, i.e., are capable of forming Watson-Crick base pairs. These terms also include the pairing of nucleoside analogs, such as deoxyinosine, nucleosides with 2-aminopurine bases, and the like, that may be employed. The term “complementary strand” is used herein interchangeably with the term “complement.” The complement of a nucleic acid strand can be the complement of a coding strand or the complement of a non-coding strand. “Essentially complimentary” refers to a duplex in which the two strands are sufficiently complimentary such as to be able to form a duplex under the desired conditions, e.g., in a cell. A mismatch in a duplex between a target polynucleotide and an oligonucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e., expression levels can be controlled).

“RNAi” stands for RNA-mediated interference.

“RNA Polymerase III promoter” or “RNA Pol III promoter” or “Pol III promoter” refers to a nucleotide sequence to which RNA Pol III can bind. Exemplary promoters include promoters of U6 snRNA, tRNAs and 5S rRNA and the H1 RNA promoter. The RNA Pol III promoter can be human, mouse, rat, drosophila or other.

The terms “polypeptide,” “peptide” and protein (when consisting of a single polypeptide chain) are used interchangeably herein when referring to a gene product. “Protein” refers to a polypeptide as well as to a molecule or complex thereof comprising two or more polypeptides, linked via disulfide bonds or not.

The term “recombinant protein” refers to a polypeptide of the present invention which is produced by recombinant DNA techniques, wherein generally, DNA encoding a protein of interest is inserted into a suitable expression vector which is in turn used to transform a host cell to produce the recombinant or heterologous protein.

“siRNA” stands for short (or small) interfering RNA.

The term “spacer” in the context of the nucleic acids of the invention refers to a nucleic acid that essentially forms the loop in the hairpin structure of an RNA transcribed from a nucleic acid of the invention.

The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

“A therapeutically effective amount” of a compound is an amount which results in a therapeutic effect in the subject to whom it was administered.

“Transcriptional regulatory sequence” is a generic term used throughout the specification to refer to DNA sequences, such as initiation signals, enhancers, promoters, and polymerase termination signals that induce or control transcription of protein coding sequences with which they are operably linked.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Exemplary Compositions

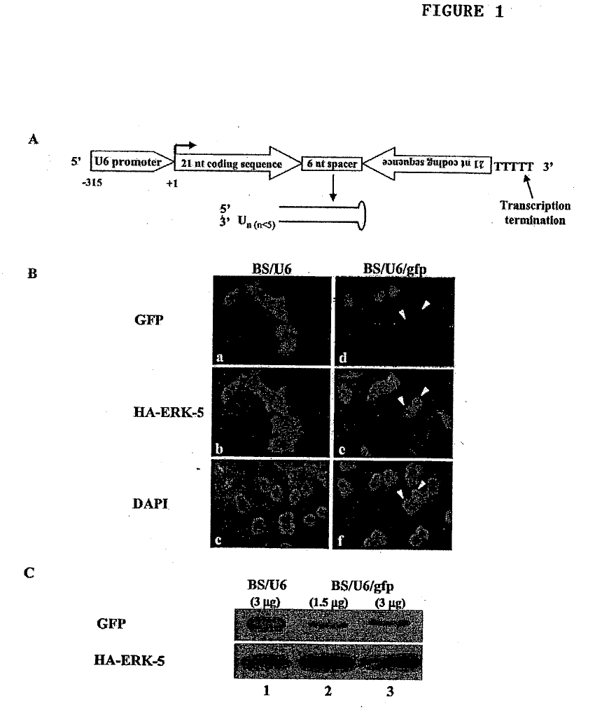

In one embodiment, the invention provides nucleic acids comprising the following nucleotide sequences in a 5′ to 3′ order: an RNA polymerase promoter sequence; a first target sequence; a spacer sequence; a second target sequence that is essentially complementary to the first target sequence; and an RNA polymerase termination signal, wherein an RNA transcribed from the nucleic acid can inhibit expression of a nucleic acid comprising a sequence that is essentially complementary to the first or the second target sequence. Gene expression may be inhibited by at least about 10%, 25%, 50%, 60%, 70%, 80%, 90%, 95% or 99%. The RNA transcribed from the nucleic acid may form a hairpin structure, wherein the first and the second target sequences hybridize to each other to essentially form the stem of the hairpin and the spacer essentially forms a loop at the closed end of the hairpin. Such hairpin RNAs may be processed in vivo by an enzyme, e.g., the Rnase III endonuclease, Dicer, into siRNAs or molecules similar thereto that are capable of inhibiting expression of specific genes. However, the RNA encoded by the nucleic acid may inhibit specific gene expression without forming a hairpin structure. An exemplary nucleic acid is shown in FIG. 1A. The nucleic acid can be DNA, or a variant of naturally-occurring DNA, such as DNA that is more resistant to degradation, provided that the nucleic acid can be transcribed into RNA.

In one embodiment, the first target sequence is sufficiently similar, e.g., essentially complementary, to a sequence of a target nucleic acid, e.g., a target gene, or the complement thereof, that the RNA transcribed from the nucleic acid is capable of inhibiting expression (e.g., specifically inhibiting expression) of the target nucleic acid. Inhibition of gene expression may be by degradation of RNA transcribed from the target gene, as siRNAs are understood to function, or by inhibition of translation, as micro RNAs (miRNAs) are understood to function. In a preferred embodiment, the first target sequence comprises a sequence that is at least about 90%, preferably at least about 95%; 97%; 98% and most preferably at least about 99% identical to a sequence of a target nucleic acid or the complement thereof. Accordingly, in some embodiments, the first target sequence differs from a sequence in a target nucleic acid in at most 5, 4, 3, 2 or 1 nucleotide. In a preferred embodiment, the first target sequence is identical to a sequence of a target nucleic acid or the complement thereof.

The first target sequence may have a length that is suitable for inhibiting gene expression. For example, the length may be suitable for the formation of a short hairpin RNA. In a preferred embodiment, the length is chosen such it is sufficient to induce gene-specific suppression, but short enough to evade a host interferon response. For example, the first target sequence may be from about 15 to about 29 or 30 consecutive nucleotides; from about 19 or 20 to about 25 consecutive nucleotides; from about 19 or 20 to 23 consecutive nucleotides long; or about 20, 21, 22 or 23 consecutive nucleotides.

The first target sequence may be essentially complementary to the coding or a non-coding portion, or combination thereof, of a target nucleic acid or complement thereof. For example, the first target sequence may be essentially complementary to the 5′ or 3′ untranslated region, promoter, intron or exon of a target nucleic acid or complement thereof. It can also be essentially complementary to a region encompassing the border between two such gene regions. In one embodiment, the first target sequence is essentially complementary to a sequence of a target gene that is located about 100 to about 200 nucleotides away, e.g., from, e.g., downstream of, the translational initiation sequence AUG.

The nucleotide base composition of the first target sequence can be about 50% adenines (As) and thymidines (Ts) and 50% cytidines (Cs) and guanosines (Gs). Alternatively, the base composition can be at least 50% Cs/Gs, e.g., about 60%, 70% or 80% of Cs/Gs. Accordingly, the choice of first target sequence may be based on nucleotide base composition. Regarding the accessibility of target nucleic acids by short RNAs, such can be determined, e.g., as described in Lee et al. (2002) Nature Biotech. 19:500. This approach involves the use of oligonucleotides that are complementary to the target nucleic acids as probes to determine substrate accessibility, e.g., in cell extracts. After forming a duplex with the oligonucleotide probe, the substrate becomes susceptible to RNase H. Therefore, the degree of RNase H sensitivity to a given probe as determined, e.g., by PCR, reflects the accessibility of the chosen site, and may be of predictive value for how well a corresponding small RNA would perform in inhibiting transcription from this target gene. One may also use algorithms identifying primers for polymerase chain reaction (PCR) assays or for identifying antisense oligonucleotides for identifying first target sequences.

First target sequences are also preferably sequences that are not likely to significantly interact with sequences other than the target nucleic acid or complement thereof. This can be confirmed by, e.g., comparing the chosen sequence to the other sequences in the genome of the target cell. Sequence comparisons can be performed according to methods known in the art, e.g., using the BLAST algorithm, further described herein. Of course, small scale experiments can also be performed, e.g., as described in the Examples, to confirm that a particular first target sequence is capable of specifically inhibiting expression of a target nucleic acid.

The second target sequence is preferably sufficiently similar (or identical) to the complement of the first target sequence, such that an oligonucleotide comprising the first target sequence and an oligonucleotide comprising the second target sequence would form a duplex in particular conditions, such as in a cell. Degrees of similarities that are sufficient for duplex formation in particular conditions are known in the art. The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases. For example, the second target sequence may comprise a nucleotide sequence which is at least about 90%, preferably at least about 95%, 97%, 98%, 99% identical to at least part of the complement of the first target sequence. Accordingly, the first and the second target sequences may differ in at most 5, 4, 3, 2 or 1 nucleotides. In a preferred embodiment, the first target sequence is perfectly complimentary to the second target sequence. The second target sequence does not have to be of the same length as the first target sequence, however, it is preferred that they be of the same length. For example, one or the other target sequence may have one or more additional nucleotides at the 5′ or 3′ end.

The target nucleic acid can be any nucleic acid, e.g., a gene, of interest whose sequence is known or can be determined. For example, as further described herein, the target nucleic acid can be a gene that is associated with a disease, e.g., cancer. It can also be a gene whose expression one may want to decrease or shutoff to reduce the likelihood of an immune rejection. Sequences of target nucleic acids of interest can be obtained from the literature and from databases, e.g., GenBank. The sequence chosen for the first target sequence is preferably, but does not always have to be, essentially complementary to the sequence of a target nucleic acid of the same species as that of the cell in which one desires to inhibit gene expression.

The spacer sequence can be any combination of nucleotides and any length provided that two complimentary oligonucleotides linked by a spacer having this sequence can form a hairpin structure, wherein at least part of the spacer forms the loop at the closed end of the hairpin. For example, the spacer sequence can be from about 3 to about 20 nucleotides; from about 5 to about 15 nucleotides; from about 5 to about 10 nucleotides; from about 3 to about 9, or about 6 nucleotides long. The sequence can be any sequence, provided that it does not interfere with the formation of a hairpin structure. In particular, the spacer sequence is preferably not a sequence having any significant homology to the first or the second target sequence, since this might interfere with the formation of a hairpin structure. The spacer sequence is also preferably not similar to other sequences, e.g., genomic sequences of the cell into which the nucleic acid will be introduced, since this may result in undesirable effects in the cell. The spacer sequence may be, or comprise a palindromic sequence. An exemplary spacer sequence is provided in the Examples and consists of the sequence AACGTT (Hind III restriction site).

The RNA polymerase can be any polymerase that is capable of transcribing relatively short DNA stretches into RNA. For example, the RNA polymerase can be RNA Polymerase II (RNA Pol II), e.g., a viral polymerase, such as the cytomegalovirus (CMV) promoter. A CMV promoter can be obtained, e.g., by PCR amplification of CMV using the following primers: 5′ AAGGTACCAGATCTTAGTTATTAATAGTAATCAATTACGG 3′ (SEQ ID NO: 1) and 5′ GAATCGATGCATGCCTCGAGACGGTTCACTAAACCAGCTCTGC 3′ (SEQ ID NO: 2), as desribed, e.g., in Xia et al. (2002) Nature Biotechnology 20: 1006. In this case, the polymerase termination signal may be a polyA sequence. In a preferred embodiment, the RNA polymerase is RNA Polymerase III (RNA Pol III). Pol III has the advantage of directing the synthesis of small, non-coding transcripts that are not capped or polyadenylated at the 5′ and 3′ ends, respectively. In addition, Pol III initiates transcription at defined nucleotides, and terminates transcription when it encounters a stretch of 4-5 thymidines (Ts). These properties make it possible to use DNA templates to synthesize in vivo, small RNAs for inhibiting gene expression, e.g., having structural features close to what has been found to be required for active siRNAs synthesized in vitro. Indeed, biochemical studies have shown that siRNAs generated by RNase III (Dicer) in Drosophila embryonic extracts contain 3′ overhangs of 2-3 nucleotides (Zamore et al. (2000) Cell 101:25; Elbashir et al. (2001) Embo J. 20:6877 and Elbashir et al. (2001) Genes & Dev. 15:188). This structural feature appears to be important for the in vitro synthesized siRNAs to effectively inhibit gene expression in cultured mammalian cells (Elbashir et al. (2001) Nature 411:494). Furthermore, siRNAs with 3′ overhangs of 2 uridines (Us) have been found to be more efficient than those with 3′ overhangs of AA, CC or GG (Elbashir et al. (2001) Embo J. 20:6877). Pol III allows the design of small RNAs which carry 3′ overhangs of one to four uridines, a structural feature close to what has been defined in vitro for effective siRNAs (Elbashir et al. (2001) Genes & Dev. 15:188). It should be noted that, although the requirements for producing small RNAs inhibiting gene expression, as described herein, may have similar requirements as those identified for siRNAs, the requirements are not identical. For example, longer overhangs on the RNA molecule than those found to be ideal for siRNAs have been shown not to affect the efficiency of gene expression inhibition.

Exemplary RNA Pol III promoters include promoters of U6 snRNA, tRNAs and 5S rRNA. Another RNA Pol III promoter that can be used is the promoter of the H1 RNA, the RNA component of nuclear RNase P (Myslinski et al. Nucleic Acids Res Jun. 15, 2001;29(12):2502). The U6 and the H1 promoter initiate transcription at G and A, respectively. Thus, the first nucleotide downstream of the promoter in a nucleic acid of the invention is preferably a G when the promoter is a U6 promoter and an A when the promoter is an H1 promoter. These nucleotides may or may not be part of the first target sequence, that is, that may or may not be part of the portion of the target nucleic acid or complement thereof.

In an illustrative embodiment, a U6 promoter is a mouse U6 promoter, e.g., comprising or consisting of nucleotides −315 to +1 (Kunkel et al. (1986) PNAS 83:8575) or a portion thereof sufficient for transcription. The nucleotide sequence of this murine U6 promoter is as follows: 5′GATCCGACGCCGCCATCTCTAGGCCCGCGCCGGCCCCCTCGCACAGACTTGTGGG AGAAGCTCGGCTACTCCCCTGCCCCGGTTAATTTGCATATAATATTTCCTAGTAACTA TAGAGGCTTAATGTGCGATAAAAGACAGATAATCTGTTCTTTTTAATACTAGCTACA TTTTACATGATAGGCTTGGATTTCTATAAGAGATACAAATACTAAATTATTATTTTAA AAAACAGCACAAAAGGAAACTCACCCTAACTGTAAAGTAATTGTGTGTTTTGAGAC TATAAATATCCCTTGGAGAAAAGCCTTGTTT(G) 3′ (SEQ ID NO: 3). The last G is the first nucleotide that is transcribed. A human U6 promoter is described, e.g., in Kunkel and Pederson (1988) Genes Dev. 2, 196-204 and Kunkel et al. Nucleic Acids Res 1989 17(18):7371 and has the following sequence: 5′AAGGTCGGGCAGGAAGAGGGCCTATTTCCCATGATTCCTTCATATTTGCATATACG ATACAAGGCTGTTAGAGAGATAATTAGAATTAATTTGACTGTAAACACAAAGATATT AGTACAAAATACGTGACGTAGAAAGTAATAATTTCTTGGGTAGTTTGCAGTTTTTAA AATTATGTTTTAAAATGGACTATCATATGCTTACCGTAACTTGAAAGTATTTCGATTT CTTGGCTTTATATATCTTGTGGAAAGGACGAAACACCG 3′ (SEQ ID NO: 4; part of GenBank Accession No. X07425). The putative TATA box of this promoter is located at nucleotides 237-242 of SEQ ID NO: 4.

Human H1 promoter is described, e.g., in Hannon et al. (1991) J. Biol. Chem. 266: 22796-22799 and GenBank Accession No. S68670 and has the following sequence: 5′ATTTGCATGTCGCTATGTGTTCTGGGAAATCACCATAAACGTGAAATGTCTTTGGA TTTGGGAATCTTATAAGTTCTGTATGAGACCACTCTTTCCCATAGGGCGGAGGGAAG CTCATCAGTGGGGCCACGAGCTGAGTGCGTCCTGTCACTCCACTCCCATGTCCCTTG GGAAGGTCTGAGACTAGGGCCAGAGGCGGCCCTAACAGGGCTCTCCCTGAGCTTCA GGGAGGTGAGTTCCCAGAGAACGGGGCTCCGCGCGAGGTCAGACTGGGCAGGAG ATGCCGTGGACCCCGCCCTTCGGGGAGGGGCCCGGCGGATGCCTCCTTTGCCGGAG CTTGGAACAGACTCACGGCCAGCGAAGTGAGTTCAATGGCTGAGGTGAGGTACCCC GCAGGGGACCTCATAACCCAATTCAGACCACTCTCCTCCGCCCATTTTTGGAAAAAA AAAAAAAAACAAAACGAAACCGGGCCGGGCGCGGTGGTTCA 3′ (SEQ ID NO: 5).

The entire sequence of a promoter provided here, or a portion thereof, can be used in the methods of the invention. Nucleic acids comprising promoter sequences can be obtained from genomic DNA of the desired species according to methods known in the art, e.g., PCR using specific primers. For example, a mouse U6 promoter can be isolated by PCR using the following primers: 5′ CCCAAGCTTATCCGACGCCGCCATCTCTA 3′ (SEQ ID NO: 6) and 5′ GGGATCCGAAGACCACAAACAAGGCTTTTCTCCAA 3′ (SEQ ID NO: 7).

Other promoters include promoters of adenovirus virus associated RNAs and promoters described in Medina et al. Curr Opin Mol Ther 1999 October;1(5):580. RNA Pol III promoters can also be made inducible, as described, e.g., in Meissner et al. (2001) Nucl. Acids Res. 29:1672. Other strategies for making a PolIII promoter inducible include inserting one or more Tet inducible promoter element, e.g., a tet operator sequence, in the PolIII promoter, e.g., between the TATA box and the proximal promoter element, as described, e.g., in Ohkama et al. (2002) Human Gene Therapy 11:577. The tet inducible system is described, e.g., in U.S. Pat. Nos. 5,654,168 and 5,650,298. The tet operator sequences can be from any class, e.g., class A, B, C, D, and E, e.g., 5′ ACTTTATCACTGATAAACAAACTTATCAGTGATAAAGA 3′ (SEQ ID NO: 8); 5′ ACTCTATCATTGATAGAGTTCCCTATCAGTGATAGAGA 3′ (SEQ ID NO: 9); 5′ AGCTTATCATCGATAAGCTAGTTTATCACAGTTAAATT 3′ (SEQ ID NO: 10); 5′ ACTCTATCATTGATAGGGAACTCTATCAATGATAGGGA 3′ (SEQ ID NO: 11); and 5′ AATCTATCACTGATAGAGTACCCTATCATCGATAGAGA 3′ (SEQ ID NO: 12). In other embodiments, the “reverse” Tet system is used (see, e.g., U.S. Pat. No. 6,271,348). Other inducible systems include the RU486-based system, which is described, e.g., in WO 93/23431 and WO 98/18925 and the ecdysone/RXR-based system that is described, e.g., in WO 96/37609 and WO 97/38117.

Yet another method for rendering promoters inducible include the insertion of a long irrelevant sequence, e.g., a 1 or 2 kb sequence surrounded by loxP (Cre-Lox) sites or other recombination facilitating sequences, e.g., Chi sites, that disrupt the promoter, and thereby render it inactive. The introduction of Cre recombinase would then render the promoter active by removing the irrelevant sequence from the promoter. LoxP/Cre system is known in the art and the sequences are publicly available (see, e.g., U.S. Pat. No. 4,959,317; Hoess et al., 1982 Proc. Natl. Acad. Sci. USA, 79:3398-3402; Sternberg et al., 1986 J. Mol. Bio., 187:197-212); Sauer and Henderson, Nature (1989) 298:447-451; and R. H. Hoess and K. Abremski, Proc. Natl. Acad. Sci., 81: 1026-1029 (1984)). Exemplary loxP sites have nucleotide sequences 5′ACTTCGTATAGCATACATTATACGAAGTTATA 3′ (SEQ ID NO: 13) and 5′ATAACTTCGT ATAATGTATACTATACGAAGTTAT 3′ (SEQ ID NO: 14). Any other site-directed homologous recombination DNA binding sites are suitable for providing an inducible promoter. These include, for example, the FRT sites of the FLP-FRT system (Schlake and Bode Biochemistry (1994) 33(43):12746-51); Rs sites of the R-Rs system (Maeser and Kahmann, 1991 Mol. Gen. Genetics, 230:170-176); the Gin-gix recombinase system of phage Mu (Klippel et al. EMBO J 1993 12(3):1047-57 and Onouchi et al., 1995 Mol. Cell. Biol., 247:653-660); and the Pin recombinase system of E. coli.

Accordingly, the invention provides nucleic acids comprising a polymerase promoter sequence and an inducible element, e.g., one of the sequences described above, that may be located within the polymerase promoter sequence, upstream or downstream of it.

The polymerase termination signal is a signal that instructs the RNA polymerase to stop transcription. In the case of Pol III polymerases, the termination signal consists of several consecutive thymidines that are transcribed into several consecutive uridines, after which the enzyme stops transcribing the template DNA. Accordingly, the second target sequence in the nucleic acid is preferably followed by at least 2, 3, 4, 5 or more consecutive thymidines.

The various sequences comprised in the nucleic acid of the invention may be directly linked to each other. Alternatively there may be one or more nucleotides between some of these sequences, e.g., nucleotides that are part of restriction recognition sequences that were used for creating the nucleic acid.

The nucleic acid of the invention may be linked to one or more additional nucleic acids. For example, it may be part of a plasmid or a vector, such as an expression vector. An expression vector can be a eukaryotic, e.g., mammalian expression vector, e.g., comprising sequences necessary for selection of cells having incorporated the vector. The vector can be integrated into the genomic DNA of a cell or it can be maintained episomally. Vectors include adenoviral vectors, and others further described herein. The expression systems can be inducible or constitutive. Accordingly, the nucleic acid may further comprise sequences necessary for replication in bacterial cells and selection of bacterial cells containing the nucleic acid, e.g., genes encoding resistance to antibiotics and an origin of replication.

Transcription of a nucleic acid of the invention may produce RNAs comprising the first and the second target sequences, the spacer sequence and at least part of the polymerase termination signal. These RNAs are expected to form hairpin structures, wherein the first and the second target sequences hybridize to essentially form the stem of the hairpin and the spacer sequence corresponds essentially to the loop at the closed end of the hairpin structure. In some embodiments, the hairpins contain about 19-29 nucleotides stems that are essentially complementary to target nucleic acid sequences; about 3-9 nucleotide loops and 3′ overhangs of five or fewer uridines. It is believed that these hairpin RNAs are processed by Dicer into active siRNAs in vivo, which then likely target RNA substrates for degradation. The small RNAs generated could, of course, also inhibit gene expression at the level of translation, similar to the action of micro RNAs (miRNAs). It is likely that small RNAs generated as described herein which are perfectly complementary to the target DNA may inhibit gene expression by targeting the RNAs for degradation, whereas small RNAs which are not perfectly complementary to the target DNA may inhibit gene expression by inhibiting translation (Ambros et al. (2001) Cell 107:823 and Gaudilliere et al. (2002) J. Biol. Chem. (September 13) ahead of print).

Accordingly, also within the scope of the invention are RNA molecules transcribed from the nucleic acids of the invention. The RNA molecules may comprise the following nucleotide sequences in a 5′ to 3′ order: a first target sequence of about 15 to about 29 or 30 nucleotides or about 19 to about 25 nucleotides, that is essentially complementary to, e.g., 95% identical to, a sequence of the target nucleic acid or the complement thereof; a spacer sequence of about 5 to 10 nucleotides; a second target sequence of about 15 to about 29 or 30 nucleotides or 19 to about 25 nucleotides that is essentially complementary to the first target sequence; and at least a portion of an RNA polymerase termination signal. The RNA may form a hairpin structure. The RNA may comprise a first and a second target sequences consisting of about 19 to about 23 nucleotides that are perfectly complementary to each other; wherein the first target sequence is perfectly complementary to a portion of a target nucleic acid or complement thereof; and at least 2 consecutive uridines at the 3′ end.

The invention further provides nucleic acids comprising the following nucleotide sequences in a 5′ to 3′ order: an RNA polymerase promoter sequence; a first restriction enzyme recognition sequence; a spacer sequence; a second restriction enzyme recognition sequence; and an RNA polymerase termination signal, wherein an RNA molecule transcribed from the nucleic acid in which a first and a second target sequences are inserted in the first and second restriction enzyme recognition site, respectively, can inhibit gene expression. Such a nucleic acid can be used to insert a first and a second target nucleic acid sequence of choice, e.g., at the restriction recognition sites. The polymerase promoter is preferably a Pol III promoter and the polymerase termination signal is preferably a stretch of 2, 3, 4, 5 or more thymidines. The nucleic acid may further comprise at least one additional restriction enzyme recognition sequence between the polymerase promoter and the first restriction enzyme recognition sequence and/or between the second restriction enzyme recognition sequence and the polymerase termination signal.

The nucleic acids of the invention may be provided in the form of a kit, optionally comprising instructions for use. The kit can further comprise one or more reagents that can be used for introducing the nucleic acids into cells, e.g., a buffer or a liposome composition or reagents to form a liposome composition. The kit may also comprise control nucleic acids, e.g., a nucleic acid of the invention for targeting the expression of a specific gene, e.g., GAPDH.

Exemplary Methods

In one embodiment, the invention provides a method for producing RNA molecules that inhibit expression of a target gene in a target cell, comprising (i) providing a nucleic acid comprising the following nucleotide sequences in a 5′ to 3′ order: an RNA polymerase promoter sequence; a first target sequence that is essentially complementary to a sequence of the target gene or complement thereof; a spacer sequence; a second target sequence that is essentially complementary to the first target sequence; and an RNA polymerase termination signal, wherein an RNA transcribed from the nucleic acid can inhibit expression of the target gene; and (ii) introducing into a target cell the nucleic acid of (i), such that the nucleic acid is transcribed in the cell and produces RNA molecules. The cell may be a eukaryotic cell, such as a mammalian cell, e.g., a human cell. The method may also be used to inhibit gene expression in lysates, e.g., cell lysates.

The invention provides methods for regulating gene expression in cells, by, e.g., degrading target RNA molecules or preventing their translation. In one embodiment, the invention provides a method for inhibiting the synthesis of a target protein in a target cell, comprising introducing into a target cell a nucleic acid comprising the following nucleotide sequences in a 5′ to 3′ order: an RNA polymerase promoter sequence; a first target sequence that is essentially complementary to a sequence of the gene encoding the target protein or complement thereof; a spacer sequence; a second target sequence that is essentially complementary to the first target sequence; and an RNA polymerase termination signal, such that the nucleic acid is transcribed in the target cell and thereby inhibits the synthesis of the target protein.

The invention also provides methods for preparing a nucleic acid for inhibiting the synthesis of a target protein in a cell, e.g., a eukaryotic cell. The method may comprise (i) providing a nucleic acid comprising the following nucleotide sequences in a 5′ to 3′ order: an RNA polymerase promoter sequence; a first restriction enzyme recognition sequence; a spacer sequence; a second restriction enzyme recognition sequence; and an RNA polymerase termination signal, wherein an RNA molecule transcribed from the nucleic acid in which a first and a second target sequences are inserted in the first and second restriction enzyme recognition site, respectively, can inhibit expression of a target nucleic acid; and (ii) introducing into the first restriction recognition sequence a first oligonucleotide of about 15-30 nucleotides comprising a sequence that is essentially complimentary to that of a target nucleic acid. The method may further comprise introducing into the second restriction recognition sequence a second oligonucleotide of about 15-30 nucleotides that is essentially complementary to the first target sequence.

Various methods for introducing nucleic acids into cells are known in the art. Exemplary methods include transfection, e.g., with calcium phosphate; electroporation; liposome based techniques, and use of viral vectors. Any type of plasmid and vector commonly used in the art can be used with the method of the invention. A plasmid that can be used may include elements that are necessary for replication of the plasmid in prokaryotic cells and elements that are necessary for selection of those prokaryotic cells including the plasmid with an insert relative to those that do not include a plasmid and those which contain an empty plasmid. In a preferred embodiment, BlueScript (BS) is used.

Any means for the introduction of the nucleic acids into cells, e.g., mammalian cells, may be adapted to the practice of this invention for the delivery of the various nucleic acids or constructs of the invention into the target cell. It may be desirable to introduce at least 5, 10, 25, 50, 100, or more copies of the nucleic acid of the invention. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. A colloidal system may be a lipid-complexed or liposome-formulated DNA. Formulation of DNA, e.g., with various lipid or liposome materials, may then be effected using known methods and materials and delivered to the recipient cell or mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Nucleic acids of the invention can also be delivered via nanotechnology.

In a preferred method of the invention, the nucleic acids are delivered using viral vectors. The nucleic acids may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of nucleic acids, e.g., exogenous genes, in vivo, particularly into humans. The following additional guidance on the choice and use of viral vectors may be helpful to the practitioner. As described in greater detail below, such embodiments of the subject expression constructs are specifically contemplated for use in various in vivo and ex vivo gene therapy protocols.

A. Adenoviral Vectors

A viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. In contrast to retrovirus, the infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect, e.g., virtually all epithelial cells regardless of their cell cycle stage. So far, adenoviral infection appears to be linked only to mild disease such as acute respiratory disease in the human.

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them preferred mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584).

Adenovirus vectors have also been used in vaccine development (Grunhaus and Horwitz (1992) Siminar in Virology 3:237; Graham and Prevec (1992) Biotechnology 20:363). Experiments in administering recombinant adenovirus to different tissues include trachea instillation (Rosenfeld et al. (1991); Rosenfeld et al. (1992) Cell 68:143), muscle injection (Ragot et al. (1993) Nature 361:647), peripheral intravenous injection (Herz and Gerard (1993) Proc. Natl. Acad. Sci. U.S.A. 90:2812), and stereotactic inoculation into the brain (Le Gal La Salle et al. (1993) Science 254:988).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 10⁹-10¹¹ plaque-forming unit (PFU)/ml, and they are highly infective. The life cycle of adenovirus does not require integration into the host cell genome. The foreign genes delivered by adenovirus vectors are episomal, and therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted nucleic acid of the invention can be under control of, for example, the E1A promoter, the major late promoter (MLP) and associated leader sequences, the viral E3 promoter, or exogenously added promoter sequences.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the method of the present invention. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the polynucleotide or construct of the invention (also referred to as “nucleic acid of interest”) in a region within the adenovirus sequences is not critical to the present invention. For example, it may also be inserted in lieu of the deleted E3 region in E3 replacement vectors as described previously by Karlsson et. al. (1986) or in the E4 region where a helper cell line or helper virus complements the E4 defect.

A preferred helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J.General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Adenoviruses can also be cell type specific, i.e., infect only restricted types of cells and/or express a nucleic acid of the invention only in restricted types of cells. For example, the viruses comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1B.

DNA sequences of a number of adenovirus types are available from Genbank. For example, human adenovirus type 5 has GenBank Accession No.M73260. The adenovirus DNA sequences may be obtained from any of the 42 human adenovirus types currently identified. Various adenovirus strains are available from the American Type Culture Collection, Rockville, Md., or by request from a number of commercial and academic sources. A nucleic acid of the invention may be incorporated into any adenoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.; PCT/FR94/00851 (WO 95/02697) by Perrocaudet et al., PCT/US95/14793 (WO96/14061) by Wang et al.

B. AAV Vectors

Yet another viral vector system useful for delivery of the subject polynucleotides is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector.

AAV is also one of the few viruses that may integrate its DNA into non-dividing cells, e.g., pulmonary epithelial cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

The AAV-based expression vector to be used typically includes the 145 nucleotide AAV inverted terminal repeats (ITRs) flanking a restriction site that can be used for subcloning of the nucleic acid of the invention, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A nucleic acid of this invention can similarly be included in an AAV-based vector. As an alternative to inclusion of a constitutive promoter such as CMV to drive expression of the polynucleotide of interest, an AAV promoter can be used (ITR itself or AAV p5 (Flotte, et al. J. Biol.Chem. 268:3781-3790, 1993)).

Such a vector can be packaged into AAV virions by reported methods. For example, a human cell line such as 293 can be co-transfected with the AAV-based expression vector and another plasmid containing open reading frames encoding AAV rep and cap (which are obligatory for replication and packaging of the recombinant viral construct) under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the nucleic acid of the invention) and expression of the viral capsid proteins. This system results in packaging of the DNA of the invention into AAV virions (Carter, B. J., Current Opinion in Biotechnology 3:533-539, 1992; Kotin, R. M, Human Gene Therapy 5:793-801, 1994)). Typically, three days after transfection, recombinant AAV is harvested from the cells along with adenovirus and the contaminating adenovirus is then inactivated by heat treatment.

Methods to improve the titer of AAV can also be used to express the nucleic acid of the invention in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997. In yet another method for producing large amounts of recombinant AAV, a chimeric plasmid is used which incorporate the Epstein Barr Nuclear Antigen (EBNA) gene, the latent origin of replication of Epstein Barr virus (oriP) and an AAV genome. These plasmids are maintained as a multicopy extra-chromosomal elements in cells, such as in 293 cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug. 19, 1997). Additionally, one may increase the efficiency of AAV transduction by treating the cells with an agent that facilitates the conversion of the single stranded form to the double stranded form, as described in Wilson et al., WO96/39530.

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J.Biol. Chem. 268:3781-3790, 1993) or chromatographic purification, as described in O'Riordan et al., WO97/08298.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

For additional detailed guidance on AAV technology which may be useful in the practice of the subject invention, including methods and materials for the incorporation of a nucleic acid of the invention, the propagation and purification of the recombinant AAV vector containing the nucleic acid of the invention, and its use in transfecting cells and mammals, see e.g. Carter et al, U.S. Pat. No. 4,797,368 (Jan. 10, 1989); Muzyczka et al, U.S. Pat. No. 5,139,941 (Aug. 18, 1992); Lebkowski et al, U.S. Pat. No. 5,173,414 (Dec. 22, 1992); Srivastava, U.S. Pat. No. 5,252,479 (Oct. 12, 1993); Lebkowski et al, U.S. Pat. No. 5,354,678 (Oct. 11, 1994); Shenk et al, U.S. Pat. No. 5,436,146(Jul. 25, 1995); Chatteree et al, U.S. Pat. No. 5,454,935 (Dec. 12, 1995), Carter et al WO 93/24641 (published Dec. 9, 1993), and Natsoulis, U.S. Pat. No. 5,622,856 (Apr. 22, 1997). Further information regarding AAVs and the adenovirus or herpes helper functions required can be found in the following articles: Bems and Bohensky (1987), “Adeno-Associated Viruses: An Update”, Advanced in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does not require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

C. Hybrid Adenovirus-AAV Vectors

Hybrid Adenovirus-AAV vectors represented by an adenovirus capsid containing a nucleic acid comprising a portion of an adenovirus, and 5′ and 3′ ITR sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100).

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected transgene. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

D. Retroviruses

The retroviruses are a group of single-stranded RNA viruses characterized by an ability to convert their RNA to double-stranded DNA in infected cells by a process of reverse-transcription “(Coffin (1990) Retroviridae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsial proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a nucleic acid of interest, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types. Integration and stable expression require the division of host cells (Paskind et al. (1975) Virology 67:242). Tthese vectors allow selective targeting of cells which proliferate.

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10:448; Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

E. Other Viral Systems

Other viral vector systems that can be used to deliver a nucleic acid of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al.(1990) J. Virol., 64:642-650).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

The invention also provides pharmaceutical compositions, comprising a nucleic acid of the invention and a pharmaceutically acceptable carrier or excipient. Methods for preparing pharmaceutical compositions are also contemplated. For example, a method of making a pharmaceutical composition may comprise combining a particular dose of a nucleic acid of the invention with a pharmaceutically acceptable carrier, such as a buffer and/or a delivery complex.

Since in certain embodiments, the compositions of the invention will be administered via a specific device, e.g., by injection using a syringe, the invention also provides devices, e.g., syringes, comprising a composition of the invention.

Exemplary Uses

The invention provides a method for regulating gene expression in cells, e.g., eukaryotic cells. Accordingly, the method can be used, e.g., to regulate gene expression in cells of a subject, e.g., a human subject. In one embodiment, a cell or tissue is obtained from a subject, a nucleic acid of the invention is introduced into the cell or cells of the tissue ex vivo, and the cell or tissue is administered to a subject, e.g., the subject from whom the cell or tissue was obtained. For example, the method can be used to reduce or inhibit the expression of certain genes in a tissue or organ that is being transplanted, e.g., to reduce the likelihood of the occurrence of an immune response by the host against the transplanted tissue or organ. Other genes that can be suppressed include those that are involved in or contribute to graft versus host diseases. Accordingly, genes that one may want to inhibit or silence include those involved in immune responses. Exemplary genes include those involved in T and B cell recognition and activation, such as costimulatory molecules, e.g., members of the B-7 family; CD4; CD8; CD3 T cell receptor components; CD40; adhesion molecules; and ligands thereof. Other genes that can be suppressed include those encoding interleukins and cytokines and other soluble molecules, which may, e.g., contribute to the proliferation of lymphocytes. Other genes which may be downregulated according to the invention include those representing potential targets of an immune response either by the graft or by the host, e.g., alloantigens and xenoantigens. Yet other genes which can be targeted include those which may be cytotoxic to a cell and genes that modulate the phenotype of a cell, e.g., its state of differentiation or growth potential.

In a particular embodiment, the nucleic acids of the invention are used to inhibit transformation of cells, e.g., the development of malignant or benign cancer cells. For example, the nucleic acids may be designed to inhibit transformation mediated by mutated genes, e.g., oncogenes, and fusion genes resulting from chromosomal translocations. The protein may be a dominantly acting mutant. The genes may be constitutively activated tyrosine kinase fusion genes, such as those associated with leukemias, e.g., chronic myeloid leukemia. Many hematopoietic cancers are caused by dominantly acting oncoproteins encoded by fusion RNA transcripts resulting from chromosomal translocations.

CML syndromes are caused by constitutively activated tyrosine kinases. There are 6 cloned chromosomal translocations associated with the CML phenotype in humans. In each instance, the consequence of the chromosomal translocation is expression of a fusion gene containing a carboxy terminal tyrosine kinase domain, and an amino terminal oligomerization motif. Examples include the BCR/ABL; TEL/PDGFβR (Golub et al. (1994) Cell 77, 307-316.), TEL/ABL (Golub et al. (1996) Mol Cell Biology 16, 4107-16); TEL/JAK2 (Lacroniqueet al. (1997) Science 278(5341), 1309-12 and Peeters et al. (1997) Blood 90, 2535-2540); HIP1/PDGFβR (Ross et al. (1998) Blood 91, 4419-26 and Ross and Gilliland, D. G. (1999) J Biol Chem 274(32), 22328-36); and H4/PDGFβR fusions (Kulkarni et al. (2000) Cancer Res 60, 3592-8 and Schwaller et al. (2001) Blood 97, 3910-8). In each case, the tyrosine kinase is constitutively activated by the oligomerization motif; transforms hematopoietic cell lines, such as Ba/F3, to factor independent growth, and causes a myeloproliferative disease in murine bone marrow transplant models. Importantly, mutations that abrogate tyrosine kinase activity result in loss of this phenotype (see, e.g., Daley et al. (1990) Science 247, 824-830 and Carroll et al. (1996) Proc Natl Acad Sci 93, 14845-14850). Taken together, these results indicate that the fusion kinases are validated targets for therapeutic intervention.

Recently, this principle has been directly proven in BCR/ABL positive CML patients treated with an ABL selective tyrosine kinase inhibitor ST1571 (Gleevec, Imatinib). ST1571 has shown remarkable activity in stable phase CML, and even in some patients with CML that has progressed to acute myeloid leukemia (AML) (Carroll et al. (1997) Blood 90(12), 4947-52; Druker et al. (2001) N Engl J Med 344, 1038-42; Druker et al. (2001) N Engl J Med 344, 1031-7; and Theising et al. (2000) Blood 96, 3195-99). However, no patients who receive Gleevec alone are cured of their disease, and responders may develop resistance to Gleevec (Sawyers, C. L. (2001) Semin Hematol 38, 15-21). There is thus a need to develop adjunctive targeted therapies for CML as well as CML blast crisis.

Accordingly, in one embodiment, nucleic acids are designed to produce RNAs targeted at BCR/ABL; Tel/Platelet Derived Growth Factor β Receptor (PDGFβR) or other fusion genes characteristic of CML. The sequences are known in the art. The activity of the RNAs can be tested by determining whether they impair the growth of hematopoietic cell lines transformed by these fusion genes as well as in murine models of myeloproliferative disease induced by tyrosine kinase fusion genes.

Nucleic acids of the invention can also be designed to inhibit acute mycloid leukemia (AML). AML is a consequence of at least two broad classes of cooperating mutations. Class I confer a proliferative and/or survival advantage to cells, but do not affect hematopoietic differentiation, and are exemplified by BCR/ABL and TEL/PDGFβR (Dash and Gilliland (2001) Best Pract Res Clin Haematol 14, 49-64 and Gilliland (2001) Curr Opin Hematol 8, 189-91). The most common of these in AML are activating mutations in the hematopoietic receptor tyrosine kinase FLT3 (Kelly et al. (2002) Blood 99, 310-8 and Kottaridis et al. (2001) Blood 98, 1752-9). Class II mutations, in contrast, result in impaired hematopoictic differentiation, and are exemplified by fusion genes involving hematopoietic transcription factors, such as the NUP98/HOXA9, AML1/ETO or PML/RARalpha fusions. AML is hypothesized to be the consequence of cooperation between these two broad classes of mutations (Dash and Gilliland (2001) Best Pract Res Clin Haematol 14, 49-64 and Gilliland (2001) Curr Opin Hematol 8, 189-91). Accordingly, the RNAs encoded by nucleic acids of the invention can be targeted to either BCR/ABL or TEL/PDGFβR or NUP98/HOXA9 for treatment of AML. Similarly, acute promyelocytic leukemia associated with a cooperation between FLT3-ITD and PML/RARalpha fusion genes can be treated by inhibiting the expression of one or both fusion proteins.

Accordingly, treatment of cancers, e.g., leukemias, may include administering to a subject in need thereof a pharmaceutically effective amount of one or more nucleic acids of the invention encoding RNAs targeted at the mutant proteins, e.g., fusion protein. The nucleic acid of the invention may be in a vector, e.g., a viral vector. Administration may be local or systemic. In cases of solid tumors, nucleic acids of the invention may be administered, e.g., by injection, directly into the tumor. In cases of leukemias, nucleic acids of the invention may be administered in organs producing the leukemic cells, e.g., into the bone marrow. Alternatively, cells may be obtained from the bone marrow, contacted ex vivo with a nucleic acid of the invention and administered back into the subject.

Other diseases that can be treated with nucleic acids of the invention include diseases that are associated with or caused by particular alleles or mutants of genes, i.e., disease associated alleles or mutants, particularly dominant, gain-of-function mutants or alleles. Single nucleotide changes can transform wild-type genes into dominant, gain-of-function mutants that cause human disease. Because the wild-type gene often performs important functions, whereas the mutant is toxic, any therapeutic strategy must selectively inhibit the mutant while retaining wild-type expression. Diseases caused by dominant, gain-of-function mutations develop in heterozygotes bearing one mutant and one wild-type copy of the gene. The best-known examples of this class are common neurodegenerative diseases, including Alzheimer's disease, Huntington's disease, Parkinson's disease and amyotrophic lateral sclerosis (ALS; “Lou Gehrig's disease”) Taylor et al. (2002) Science 296, 1991-5.

In these diseases, the exact pathways whereby the mutant proteins cause cell degeneration are not clear, but the origin of the cellular toxicity is known to be the mutant protein. Mutations in Cu, Zn superoxide dismutase (SOD1) cause motor neuron degeneration that leads to ALS, because the mutant protein has acquired some toxic property (Cleveland, D. W. & Rothstein, J. D. Nat Rev Neurosci 2, 806-19(2001)). Neither the molecular cause of this toxic property nor how the toxic protein triggers motor neuron degeneration is understood. In mice, expression of mutant SOD1, but not complete elimination of SOD1, causes ALS. Nonetheless, SOD1-knockout mice show reduced fertility, motor axonopathy, age-associated loss of cochlear hair cells and neuromuscular junction synapses, and enhanced susceptibility to a variety of noxious assaults, such as axonal injury, ischemia, hemolysate exposure and irradiation, on the nervous system (Matzuk, et al. Endocrinology 139, 4008-11. (1998); Shefner et al. Neurology 53, 1239-46 (1999); McFadden et al. Audiology 40, 313-21 (2001); Flood et al. Am J Pathol 155, 663-72 (1999); Reaume et al. Nature Genetics 13, 43-7 (1996); Kawase et al. Stroke 30, 1962-8 (1999); Kondo et al. Journal of Neuroscience 17, 4180-9 (1997); Matz et al. Stroke 31, 2450-9 (2000) and Behndig et al. Free Radic Biol Med 31, 738-44 (2001)). Given the toxicity of the mutant protein and the functional importance of the wild-type, the ideal therapy for ALS would selectively block expression of the mutant while retaining expression of wild-type protein.

The vast majority of ALS-causing SOD1 mutations are single-nucleotide point mutations that alter single amino acid in the protein (http://www.alsod.org/). Accordingly, ALS can be treated or prevented by a method comprising administering to the subject a pharmaceutically effective amount of a nucleic acid of the invention comprising a first targeting sequence that is essentially complementary, and preferably perfectly complementary, to a sequence of the SOD1 gene comprising a point mutation or complement thereof. The point mutation may be located in the middle of the first target sequence, or at 1, 2, 3, 4, 5 or more nucleotides away from the middle of the first target sequence. For example, in a first target sequence of 19 nucleotides, the mutation may be located at nucleotide 10 or at nucleotides 7, 8, 9, 11, 12 or 13. As described in the Examples and further herein, a construct comprising a particular first target sequence can be tested in vitro. First target sequences that can be used in a method for treating ALS are provided in the Examples. To illustrate, a nucleic acid comprising a first target sequence comprising or consisting of 5′ GGAGACTTGCGCAATGTGA 3′ (SEQ ID NO: 15) or the complement thereof, i.e., 5′ TCACATTGCGCAAGTCTCC 3′ (SEQ ID NO: 16) can be used for inhibiting expression of expression of a G256C (Gly85Arg) SOD1 mutant gene. A nucleic acid comprising a first target sequence comprising or consisting of 5′ GACAAAGATGCTGTGGCCGAT 3′ (SEQ ID NO: 17) or the complement thereof, i.e., 5′ ATCGGCCACAGCATCTTTGTC 3′ (SEQ ID NO: 18) can be used for inhibiting the expression of a G281C (Gly93Ala) SOD1 mutant gene.

Numerous other point mutations have been found in SOD1 in ALS patients, and all of these can also be targeted for therapy. Exemplary mutations include nucleotide 93 (Ala4Ser; Ala4Thr and Ala4Val); nucleotide 103 (Val7Glu); nucleotide 105 (Leu8Val and Leu8Gln); nucleotide 113 (Gly10Gly); nucleotide 117 (Gly12Arg); nucleotide 123 (Val14Met and Val14Gly); nucleotide 129 (Gly16Ser and Gly16Ala); nucleotide 144 (Glu21Lys and Glu21Gly); nucleotide 466 (Gly37Arg); nucleotide 469 (Leu38Val and Leu38Arg); nucleotide 478 (Gly41Ser and Gly41Asp); nucleotide 485 (His43Arg); nucleotide 491 (Phe45Cys); nucleotide 494 (His46Arg); nucleotide 501 (His48Gln); nucleotide 502 (Glu49Lys); nucleotide 1646 (Ser59Ser); nucleotide 663 (Asn65Ser); nucleotide 669 (Leu67Arg); nucleotide 683 (Gly72Ser); nucleotide 695 (Asp76Tyr and Asp76Val); nucleotide 1059 (Leu84Val and Leu84Phe); nucleotide 1062 (Gly85Arg); nucleotide 1066 (Asn86Ser); nucleotide 1075 (Ala89Val); nucleotide 1078 (Asp90Ala and Asp90Val); nucleotide 1086 (Gly93Cys, Gly93Arg, Gly93Ser, Gly93Asp and Gly93Ala); nucleotide 1092 (Ala95Thr); nucleotide 1095 (Asp96Asn); nucleotide 1098 (Val97Met); nucleotide 1107 (Glu100Lys and Glu100Gly); nucleotide 1110 (Asp101Asn and Asp101Gly); nucleotide 1119 (Ile104Phe); nucleotide 1123 (Ser105Leu); nucleotide 1125 (Leu106Val); nucleotide 1132 (Gly108Val); nucleotide 1134 (Asp109Asn); nucleotide 1144 (Ile112Thr and Ile112Met); nucleotide 1146 (Ile143Phe and Ile113Thr); nucleotide 1150 (Gly114Ala); nucleotide 1152 (Arg115Gly); nucleotide 1162 (Val118insAAAAC); nucleotide 1441 (Asp124Val); nucleotide 1443 (Asp125His); nucleotide 1446 (Leu126delTT, Leu126STOP and Leu126Ser); nucleotide 1450 (Gly127insTGGG); nucleotide 1465 (Glu132insTT); nucleotide 1467 (Glu133del); nucleotide 1471 (Ser134Asn); nucleotide 1487 (Asn139Asn and Asn139Lys); nucleotide 1489 (Ala140Gly and Ala140Ala); nucleotide 1501 (Leu144Ser and Leu144Phe); nucleotide 1503 (Ala145Thr and Ala145Gly); nucleotide 1506 (Cys146Arg); nucleotide 1512 (Val148Ile and Val148Gly); nucleotide 1516 (Ile149Thr); nucleotide 1522 (Ile151Thr); nucleotide 1529 (Gln153Gln) (see, http://www.alsod.org/). The nucleotide number refers to the position in the genomic DNA of SOD1.

ALS is also associated with mutations in genes NEFH (neurofilament, heavy polypeptide 200 kDa) and ALS2 (amyotrophic lateral sclerosis 2 (juvenile) homolog (human)). These mutations are set forth at (http://www.alsod.org/). Accordingly, ALS can also be treated by targeting these mutations using the methods described herein.

Other diseases associated with or caused by mutated proteins include hemophilia. Such diseases can be treated or prevented by administering to a subject having a gene mutation or disease associated allele a nucleic acid of the invention that is transcribed into RNA inhibiting the expression of the mutated protein or disease associated allele. For example, a nucleic acid may comprise a first target sequence that is essentially complementary to a portion of the gene encoding the mutated protein or the disease associated allele.

Pathogenic diseases, e.g., viral or bacterial diseases or infections, can also be treated or prevented by administration of nucleic acids of the invention. For example, a nucleic acid encoding RNA that can inhibit the expression of pathogenic protein that is crucial for the replication or integration of the pathogen's genome, transmission of the pathogen; maintenance of the infection; entry into a host; drug metabolism by the pathogen; can be administered to a subject. Thus, methods of prophylaxis (i.e., prevention or decreased risk of infection), as well as reduction in the frequency or severity of symptoms associated with infection are contemplated. Exemplary diseases include cervical carcinomas, which are caused by Human Papilloma Viruses (HPV), in which case the method of the invention would target a protein that is crucial for its replication or maintenance or otherwise necessary for the virus.

When used in methods for treating or preventing diseases, e.g., cancer, the nucleic acids of the invention can be administered together with other drugs, surgery or other treatment that is administered for treating the disease. Administration can be conducted simultaneously or consecutively. For example, a nucleic acid of the invention can be administered as part of a cocktail therapy, e.g., with other chemotherapeutic drugs.

In another embodiment, the nucleic acids of the invention can be used to block or reduce undesirable effects of certain drugs or toxins. For example, the number of receptors of toxins can be reduced on the cell surface, so that the entry of toxins into cells is inhibited. The cell surface receptors of pathogens can also be targeted.

Most protein exotoxins, which are produced by bacterial pathogens follow an “A-B” model. These are composed of two chains or two types of subunits, or two domains. The “A” chain or subunit contains the active portion of the toxin, while the “B” subunit is responsible for recognition of toxin receptors and internalization of the A-subunit. Accordingly, inhibition of the action of toxin can be achieved by inhibition of production of either the A subunit or the B subunit. An exemplary toxin receptor that can be targeted is the anthrax toxin receptor PA, which is necessary for the toxicity of the anthrax toxin. The nucleotide sequence of the human receptor is provided in GenBank Accession number AF421380 (homo sapiens anthrax toxin receptor mRNA). Another receptor that can be targeted is the exotoxin A receptor, which is encoded by the nucleotide sequence provided in GenBank Accession number NM_{—}002332 (homo sapiens low density lipoprotein-related protein 1 (alpha-2-macroglobulin receptor) (LRP1)).

In addition, many toxins have to be proteolytically activated during entry into the target cell by cleavage with a mammalian protease called furin. Accordingly, inhibition of the synthesis of furin inhibits the action of the toxin. Thus, a subject can be protected from the effect of such toxins by administration to the subject of a construct of the invention comprising a first target sequence that is essentially complementary to a sequence of the furin gene or complement thereof. The human furin gene (also referred to as “membrane associated receptor protein” gene or PACE) sequence is provided in GenBank Accession number NM_{—}002569.

Other toxins receptors are associated with a particular protein. For example, the toxin receptor of P. aeruginosa exotoxin A has an associated protein (RAP) that modulates internalization of the toxin. Accordingly, modulating, e.g., interfering with or inhibiting, the production of the associated protein would modulate, e.g., prevent, the toxin from being internalized. The nucleotide sequence of the gene encoding the human RAP protein is provided in GenBank Accession number NM_{—}002337 (Homo sapiens low density lipoprotein-related protein-associated protein 1 (alpha-2-macroglobulin receptor-associated protein 1) (LRPAP 1)).

Finally, the group of toxins including ADP-ribosylate elongation factor 2 (EF-2) requires to be modified for toxicity. This modification is a post-translational methylation of a single histidine on EF-2, converting the histidine to an unusual amino acid called diphthamide. The enzyme involved in this conversion is diphthamide synthase. Accordingly, the effect of this toxin can be inhibited by targeting this enzyme using constructs of the invention. The nucleotide sequence of the human enzyme is provided in GenBank Accession number NM_{—}001384 (Homo sapiens diphtheria (and exotoxin A) toxin resistance protein required for diphthamide biosynthesis-like 2 (S. cerevisiae) (DPH2L2)).

The methods described herein can also be used to identify genes that are involved in particular biological functions. For example, the nucleic acids of the invention can be used to identify genes that are involved in toxic effects on cells. Accordingly, introducing into cells nucleic acids encoding RNAs that inhibit the expression of particular proteins and treating the cells with toxic agents, will reveal which proteins are necessary for the toxic effect of these toxic agents. Based on these results, drugs that prevent expression of these proteins (e.g., nucleic acids of the invention) can be developed to protect subjects exposed to the toxic agent to be subject to the toxic effects thereof.

Other uses of the invention include the production of cells in which the expression of one or more genes is suppressed for use in producing a recombinant protein. For example, when a recombinant protein is produced in a host cell, e.g., a mammalian host cell, it may be desirable to inhibit the expression of one or more other proteins, such as proteins that are likely to be co-purified with the recombinant protein and thus, undesirable. Accordingly, a host cell is modified by the introduction into the host cell of a nucleic acid of the invention comprising a first target sequence that is essentially complementary to a portion of a target gene or complement thereof. The host cell can be modified by stable or transient transfection, such that the nucleic acid of the invention is integrated or not, into the genome of the host cell. Stable transfection or modification refers to the integration of the nucleic acid or a portion thereof into the genome of the cell or its existence in the form of an episome. For example, in one embodiment, a nucleic acid of the invention is introduced into host cells; the host cells having integrated the nucleic acid into their genome or in the form of an episome are selected; and those transformed host cells are then used to express the recombinant protein of choice. Alternatively, a nucleic acid of the invention and a nucleic acid encoding a recombinant protein of choice can be introduced essentially simultaneously into a host cell.

In yet another embodiment, the nucleic acids and methods of the invention are used to study the role of particular genes in cells. In an illustrative embodiment, when it is desired to know the effect of turning off a particular gene in a cell, a nucleic acid of the invention encoding an siRNA targeted at the particular gene is introduced into the cell, and its effect is analyzed.

The level of inhibition of gene expression resulting from expression of the RNA in cells can be monitored according to methods well known in the art and further described herein. For example, the level of a protein, whose expression is targeted, can be determined using antibodies directed against the protein.

Pharmaceutical Compositions

As discussed above, various diseases can be treated by administration to the subject of a pharmaceutically effective amount of a nucleic acid of the invention. A nucleic acid is generally administered in the form of a vector, such as a viral vector comprising all transcriptional regulatory elements necessary for appropriate expression in a target cell.

Depending on the type of disease, different methods of administration of the nucleic acids of the invention can be used. For example, a skin disease can be treated by applying a nucleic acid of the invention together with an appropriate delivery vehicle to the skin. For treating a lung disease, the nucleic acid can be inhaled. Alternatively, for treating a tissue that is inside a human body, a nucleic acid of the invention may have to be injected to the desired site. Set forth below are general guidelines for administration of nucleic acids.

The therapeutic methods of the invention generally comprise administering to a subject in need thereof, a pharmaceutically effective amount of a nucleic acid. The nucleic acid of the invention can be administered in a “growth inhibitory amount,” i.e., an amount of the nucleic acid which is pharmaceutically effective to inhibit or decrease proliferation of target cells. The nucleic acids of this invention may be administered to mammals, preferably humans, either alone or, preferably, in combination with pharmaceutically acceptable carriers, excipients or diluents, in a pharmaceutical composition, according to standard pharmaceutical practice. The nucleic acids can be administered orally or parenterally, including the intravenous, intramuscular, intraperitoneal, subcutaneous, rectal and topical routes of administration.

Toxicity and therapeutic efficacy of the nucleic acids can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Reagents which exhibit large therapeutic indices are preferred. While reagents that exhibit toxic side effects may be used, care should be taken to design a delivery system that targets such reagents to the site of affected tissue in order to minimize potential damage to normal cells and, thereby, reduce side effects.

The data obtained from cell culture assays and animal studies can be used in formulating a range of dosage for use in humans. The dosage of such reagents lies preferably within a range of circulating concentrations that include the ED₅₀ with little or no toxicity. The dosage may vary within this range depending upon the dosage form employed and the route of administration utilized. For any nucleic acid used in the method of the invention, the therapeutically effective dose can be estimated initially from cell culture assays. A dose may be formulated in animal models to achieve a circulating plasma concentration range that includes the IC₅₀ (i.e., the concentration of the nucleic acid which achieves a half-maximal inhibition of symptoms) as determined in cell culture. Such information can be used to more accurately determine useful doses in humans.

The pharmaceutical compositions containing the nucleic acid may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the nucleic acid in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions may contain the nucleic acid (i.e., active ingredient) in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant nucleic acid. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions of the invention may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the composition of the invention are employed. For purposes of this application, topical application shall include mouth washes and gargles.

The compositions for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The nucleic acids of the invention may also be co-administered with other well known therapeutic agents that are selected for their particular usefulness against the condition that is being treated.

When a composition according to this invention is administered into a human subject, the daily dosage will normally be determined by the prescribing physician with the dosage generally varying according to the age, weight, and response of the individual patient, as well as the severity of the patient's symptoms.

It may also be advantageous to administer the compound of the invention utililizing a method of a slow release, as known in the art.

The present invention is further illustrated by the following examples, which should not be construed as limiting in any way. The contents of all cited references including literature references, issued patents, published and non published patent applications and GenBank Accession numbers as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### siRNA Synthesized from DNA Templates In Vivo Efficiently Inhibit a Transfected Gene in Mammalian Cells

Tuschl and colleagues (14) defined the active, in vitro synthesized siRNA as a 21-nucleotides-long dsRNA with symmetrical 2- to 3-nucleotides 3′ overhangs (14). In other organisms such as C. elegans and Drosophila, the input RNA can be either in the form of a long dsRNA or a hairpin dsRNA (15, 16). Presumably, both forms of RNA are further cleaved by Dicer, a RNase III enzyme, to generate 21- to 23-nucleotides-long siRNA (17-19). To synthesize, from a DNA template, a small RNA displaying features close to these requirements, RNA Pol III, which directs transcription that terminates at a run of 4-5 Ts, was used, making it possible to design RNA with defined ends. The strategy adopted is shown in FIG. 1A. Briefly, DNA fragments that acted as templates for the synthesis of small RNAs were inserted under the control of the mouse U6 promoter that directs the synthesis of a Pol III-specific RNA transcript (20). The resulting RNA was composed of two identical 21-nucleotides sequence motifs in an inverted orientation, separated by a 6-bp spacer of nonhomologous sequences. Five Ts that function as a termination signal for Pol III (13) were added at the 3′ end of the repeat (FIG. 1A). This RNA is predicted to fold back to form a hairpin dsRNA with a 3′ overhang of several Ts (FIG. 1A). Although the exact structure of this small RNA is unknown, it robustly inhibited gene expression in vivo as described below. We therefore use the term siRNA to refer to these molecules, noting that the RNAs may not have the same structure or requirements for inhibiting gene expression as siRNAs have.

Previous studies showed that the length of the 3′ overhangs plays a role in determining the activity of siRNA synthesized in vitro (14). Although the exact number of Ts has not been determined, we expect the 3′ overhang of the siRNA transcribed by Pol III in our system not to exceed five Ts (FIG. 1A). This prediction is based on the fact that human Pol III stops within or immediately after the five Ts (13). This structure preserves some of the features of the siRNA defined by Tuschl and colleagues (12) but is nevertheless distinct. Notably, only one end of the siRNA is exposed and its 3′ overhang is predicted to be slightly longer than 2-3 nucleotides. Despite these differences, these siRNAs functioned effectively to inhibit gene expression in mammalian cells. We used this strategy to generate DNA templates for synthesis of siRNAs corresponding to the gfp (BS/U6/gfp), human lamin A/C (BS/U6/lamin A/C), cdk-2 (BS/U6/cdk-2), and dnmt-1 (BS/U6/dmnt-1) genes.

To determine whether this DNA vector-based approach can be used to inhibit gene expression in mammalian cells, the DNA template-derived siRNA was first tested on a transfected plasmid encoding the green fluorescent protein (GFP). A plasmid, BS/U6/gfp, that carries the U6 promoter (−315+1; SEQ ID NO: 3) linked at the +1 position to an inverted repeat matching a 21-nucleotides coding region within the gfp gene was constructed as described in Example 4. The two motifs that form the inverted repeat were separated by a spacer of 6 nucleotides. The transcriptional termination signal of five Ts was added at the 3′ end of the inverted repeat. The resulting siRNA is predicted to fold back to form a hairpin dsRNA as shown in FIG. 1A.

Either the BS/U6 (empty vector) or BS/U6/gfp vector were transfected together with the target cytomegalovirus (CMV)-GFP plasmid and an unrelated HA-ERK-5 plasmid at a ratio of 20:1 (effector versus target plasmids) into HeLa cells (100 ng of CMV-GFP and 0.5 μg of HA-ERK-5 plasmids). This ratio of effector versus target plasmid was chosen to ensure that cells that received the GFP and HA-ERK-5 target plasmids also received the RNAi (effector) plasmid.

We then assayed for GFP and HA-ERK-5 expression within the same transfected cells by immunostaining. The results, which are presented in FIG. 1B, show that, whereas vector BS/JU6 had no effect on GFP expression, BS/U6/gfp greatly diminished its expression. In most transfected cells (i.e., displaying HA-ERK-5 expression), GFP expression was reduced to near background levels as shown in FIG. 1B (two such cells are indicated by solid arrows in d). Significantly this effect is gene-specific because BS/U6/gfp did not inhibit the expression of cotransfected HA-ERK-5 (FIG. 1B, compare b and e). The presence of HA-ERK-5 did not affect RNAi because in its absence we observed similar level of inhibition of GFP by the GFP siRNA.

The immunostaining results were further confirmed by Western blotting, as shown in FIG. 1C. We used two different doses of the BS/U6/gfp plasmid in the experiment (1.5 and 3.0 μg) and observed similar reduction of GFP levels (FIG. 1C, compare lanes 2 and 3). Compared with the BS/U6 vector control, the inhibition of GFP expression by BS/U6/gfp was estimated to be more than 80% (FIG. 1C, compare lanes 2 and 3 with lane 1). The lack of complete inhibition may in part be caused by high level of GFP expression that was directed by a strong CMV promoter. As shown below, RNAi inhibition of endogenous genes appeared to be more robust.

Taken together, these findings demonstrate that the DNA vector-based RNAi approach functions in mammalian cells.

### Example 2

### Efficient Inhibition of Three Endogenous Genes by siRNAs Synthesized from DNA Templates In Vivo

This Example demonstrates that siRNA synthesized from a vector introduced into cells functions to inhibit expression of endogenous genes.

Three endogenous genes with diverse functions were used as targets. The first gene targeted for repression was the human lamin A/C gene, which has been shown to be effectively inhibited in cell culture by in vitro-synthesized siRNAs (12). We transfected HeLa cells with either BS/U6 vector or BS/U6/lamin A/C, which directs synthesis of a lamin A/C siRNA in vivo, together with CMV-GFP to mark the transfected cells (see Example 4 for details on the vectors). The presence of lamin A/C was detected with an anti-lamin A/C antibody. Cells transfected with either the vector BS/U6 or the RNAi plasmid BS/U6/lamin A/C were also stained with antibodies that recognized the related lamin B protein.

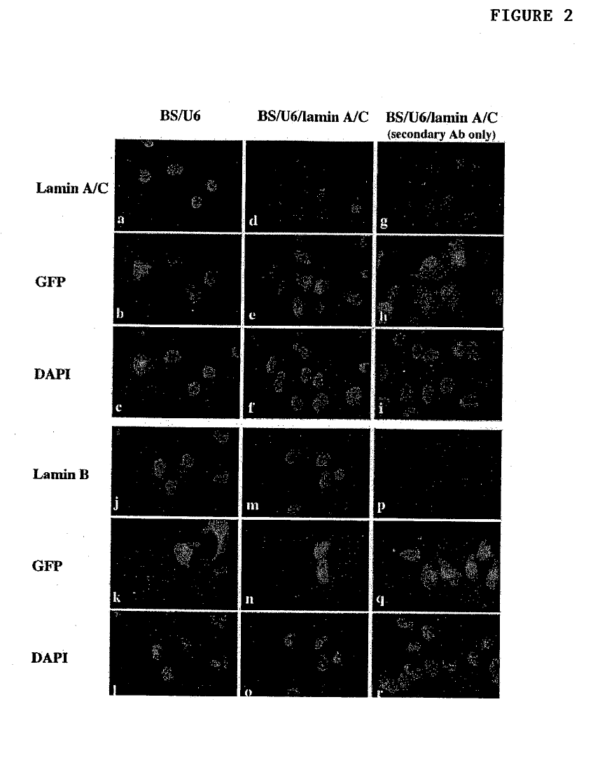

The results, which are depicted in FIG. 2, show that, whereas BS/U6 vector had no significant effect on lamin A/C expression (a), the plasmid BS/U6/lamin A/C reduced lamin A/C expression in transfected cells to levels comparable to those seen with the secondary antibody alone (compare d with a and g). Significantly, BS/U6/lamin A/C siRNA had no effect on the expression level of the related lamin B gene (FIG. 2, compare m with j), demonstrating that the observed RNAi effect is gene-specific.

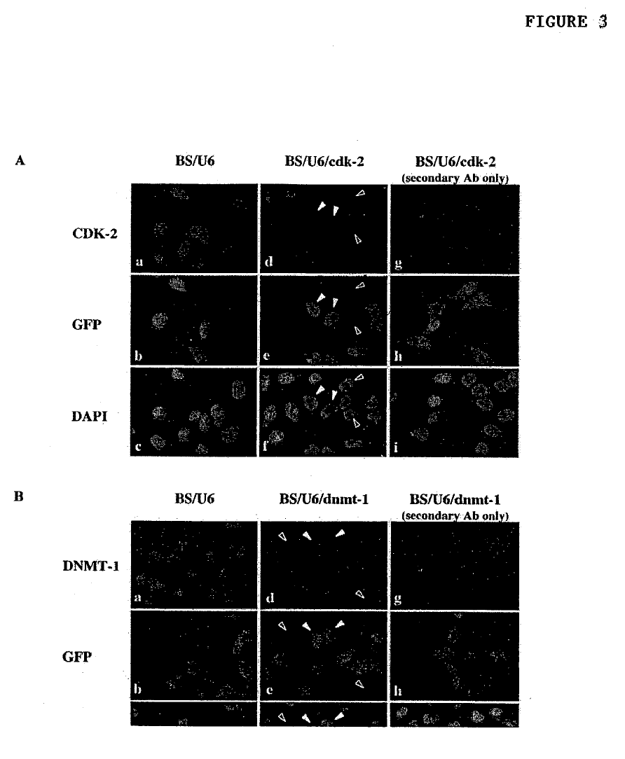

Having demonstrated that the DNA vector-based RNAi worked well to inhibit the expression of the lamin A/C gene, we asked whether this strategy functioned broadly to inhibit genes of interest by examining two additional genes involved in different aspects of cell biology. The second gene we targeted for repression was the human cyclin-dependent kinase-2 (cdk-2) gene, which plays an important role in cell cycle control (reviewed in ref. 21). We constructed a plasmid carrying a DNA template that directs the synthesis of a cdk-2 siRNA and carried out transient transfection in HeLa cells as described above. GFP was cotransfected to mark the transfected cells. As shown in FIG. 3A, cells that have been transfected with the BS/U6/cdk-2 plasmid had significantly reduced, close to background, level of CDK-2 protein compared with BS/U6 vector-transfected cells (compare panel d with a and g, two transfected cells were indicated by solid arrows). In contrast, cdk-2 expression in nontransfected cells was comparable to that observed in cells transfected with the vector control (FIG. 3Aa). Because we transfected significantly more BS/U6/cdk-2 plasmid than GFP plasmid (20:1 ratio), some of the cells may have received only the RNAi plasmid and not the GFP plasmid. As expected, some GFP-negative cells (indicated by open arrows in FIG. 3A) also had reduced CDK-2 expression near to background level (FIG. 3A, compare d with a and g).

Thus, these data indicate that cdk-2 siRNA worked efficiently to reduce CDK-2 expression in vivo.

Gene DNA methyltransferase-1 (DNMT-1), which plays an important role in maintaining patterns of CpG methylation and the epigenetic control of gene expression in mammals was also targeted (22). As shown in FIG. 3B, similar to what has been observed for lamin A/C and cdk-2, dnmt-1 expression can be efficiently inhibited by BS/U6/dnmt-1 siRNA in vivo (compare d with a). Once again, we observed GFP-negative cells (indicated by open arrows in FIG. 3B) that also had reduced DNMT-1 expression. As mentioned above, these cells may have received only BS/U6/dnmt-1 vector but not the GFP plasmid, because of the high RNAi-to-GFP plasmid ratio used for the cotransfections.

Thus, these results demonstrate that expression of endogeneous genes of mammalian cells can be efficiently inhibited (or suppressed) by expressing in the cells a vector encoding RNAs targeted to the genes.

### Example 3

### siRNAs Synthesized from DNA Templates In Vivo Inhibit Gene Expression in Different Cell Lines

This Example demonstrates that siRNAs synthesized from DNA templates in vivo can inhibit gene expression in different cell lines.

In addition to HeLa cells, the activity of the cdk-2 and lamin A/C siRNA plasmids was analyzed in three additional cell lines: H1299 (nonsmall cell lung carcinoma), C-33A (human papilloma virus negative cervical carcinoma), and U-2 OS (osteosarcoma). Cells were transfected with either BS/U6 or siRNA plasmids BS/U6/cdk-2, or BS/U6/lamin A/C together with CMV-GFP to mark the transfected cells. Cells were analyzed by immunofluorescence for endogenous CDK-2 or lamin A/C expression. For each data point, 200 GFP-positive cells were scored for the presence of CDK-2 or lamin A/C signals. The results are shown in Table 1. For each data point shown in Table 1, 200 GFP-positive cells were counted for the expression of either CDK-2 or lamin A/C. The values correspond to the percentage of GFP-positive cells that display a reduction of the CDK-2 or lamin A/C immunostaining near the background level and are presented as the average ±SD.

The results indicate that, in the presence of the BS/U6 vector, the CDK-2-negative/GFP-positive cells ranged from 0.2% to 5.2%, whereas the lamin A/C-negative/GFP-positive cells ranged from 1.2% to 4.3%. In contrast, in the presence of the cdk-2 or lamin A/C siRNA plasmid, we observed dramatic increases in the number of CDK-2 or lamin A/C-negative/GFP-positive cells (86.9% to 97.7%; and 93.5% to 95.2%, respectively, Table 1).

TABLE 1
Inhibition of cdk-2 and lamin A/C gene expression in
different human cell lines

| | % of CDK-2-negative/ | | % of Lamin A/C-negative/ | |
|---|---|---|---|---|
| | GFP-positive cells | | GFP-positive cells | |
| Cells | BS/U6 | BS/U6/cdk-2 | BS/U6 | BS/U6/lamin A/C |
| HeLa | 0.2 ± 0.4 | 97.7 ± 1.0 | 2.0 ± 0.8 | 95.2 ± 2.1 (n = 4) |
| | (n = 6) | (n = 6) | (n = 4) | |
| H1299 | 4.0 ± 0.5 | 86.9 ± 1.5 | 4.3 ± 1.7 | 93.5 ± 1.8 (n = 3) |
| | (n = 3) | (n = 3) | (n = 3) | |
| U-2 OS | 5.2 (n = 2) | 92.4 (n = 2) | 1.2 (n = 2) | 93.8 (n = 2) |
| C-33A | 3.7 (n = 2) | 92.3 (n = 2) | n.d. | n.d. |

n, number of transfection experiments.
n.d., not determined.

Thus, these results demonstrate that siRNAs synthesized from DNA templates in vivo cause robust, near complete inhibition of endogenous gene expression in a variety of cells, suggesting that this technique is generally applicable to studying gene function in mammalian cells.

Generally, these results demonstrate that a DNA vector-based RNAi approach functions effectively to silence endogenous gene expression in mammalian cells. This process is expected to greatly facilitate the use of the RNAi technology for gene function studies in mammalian cells and perhaps in vertebrate animals as well. The technology can be adapted to analyzing gene function over a long period through stable inhibition. It also can be adapted to establish an inducible siRNA system that can knock down gene expression in a regulated fashion. This latter feature is necessary for studying genes whose products are required for cell viability. Finally, the vector-based RNAi technology makes it possible to consider RNAi as a reverse genetic tool for genome level analysis of mammalian gene functions.

### Example 4

### Materials and Methods for Examples 1-3

Plasmids that contain DNA templates for the synthesis of siRNAs under the control of the U6 promoter were prepared as follows. Plasmid pmU6 containing the mouse U6 promoter (−315/+1) (S. Altman, Yale University, New Haven, Conn.) was used as a template for PCR isolation of the U6 promoter (−315 to +1) with an added ApaI cloning site at the transcriptional initiation site, which was cloned into Bluescript (BS) to generate the parent plasmid BS/U6. A general strategy for constructing an RNAi plasmid involved subcloning an inverted repeat into BS/U6 at the ApaI site. The selection of the coding sequences for siRNA was empirically determined but they started with GG and were analyzed by BLAST research to ensure that they did not have significant sequence homology with other genes. Insertion of the individual repeat motifs into BS/U6 was achieved in two separate steps. For example, to generate the BS/U6/gfp RNAi plasmid, a 22-nucleotides oligo (oligo 1) corresponding to nucleotides 106-127 of the green fluorescent protein (GFP) coding region was first inserted into the BSIU6 vector digested with ApaI (blunted) and XhoI. The inverted motif that contains the 6-nucleotides spacer and five thymidines (Ts) (oligo 2) was then subcloned into the XhoI and EcoRI sites of the intermediate plasmid to generate BS/U6/gfp. Oligo 1 is 5′-GGCGATGCCACCTACGGCAAGC-3′ (forward) (SEQ ID NO: 19) and 5′-TCGAGCTTGCCGTAGGTGGCATCGCC-3′ (reverse) (SEQ ID NO: 20). Oligo 2 is 5′-TCGAGCTTGCCGTAGGTGGCATCGCCCTTTTTG-3′ (forward) (SEQ ID NO: 21) and 5′-AATTCAAAAAGGGCGATGCCACCTACGGCAAGC-3′ (reverse) (SEQ ID NO: 22).

RNAi plasmids for the endogenous genes [lamin A/C, cyclin-dependent kinase-2 (cdk-2), and DNA methyltransferase-1 (dnmt-1)] were created essentially as described above. The sequences for the bodies of the siRNAs for lamin A/C, cdk-2, and dnmt-1 were taken from GenBank accession nos. XM-086566 (nucleotides 1627-1647), XM-049150 (nucleotides 652-672), and NM-001379 (nucleotides 598-617), respectively.

Cell culture and transfections were conducted as follows. HeLa, U-2 OS, H1299, and C-33A (American Type Culture Collection) cells were cultured in DMEM (GIBCO) supplemented with 10% of heat-inactivated FBS. Cells grown on coverslips in 6-well plates were transfected by using a calcium phosphate method and harvested 2-3 days after the transfection. Separate plasmids encoding GFP and siRNAs were generally used at a ratio of 1:10-1:30.

Immunofluorescence microscopy was conducted as follows. Cells were harvested 3 days posttransfection for analysis. They were washed once with PBS and fixed with 3% paraformaldehyde in PBS for 20 min at room temperature. The cells were permeabilized with PBS containing 0.5% of Igepal CA-630 nonionic detergent (Sigma) for 10 min and washed twice in PBS containing 0.1% of Igepal (washing buffer). After blocking with washing buffer containing 10% FBS, cells were incubated with the appropriate primary antibodies for 2-4 h at room temperature. The anti-DNMT-1, anti-CDK-2, and anti-lamin B antibodies (Santa Cruz Biotechnology) were used at the dilutions of 1/50, 1/150, and 1/100 in blocking buffer, respectively. The monoclonal anti-hemagglutinin (HA) antibody was from Babco (Richmond, Calif.), and anti-lamin A/C antibody was from Cell Signaling (Beverly, Mass.). They were used at the dilutions of 1/300 and 1/100, respectively. After three washes, the cells were incubated with the corresponding secondary antibodies for 30 min at room temperature and washed three times with the washing buffer and once with PBS. The coverslips were then rapidly rinsed in water before being mounted in Vectashield medium (Vector Laboratories). The coverslips were analyzed by fluorescence microscopy (Leica, Deerfield, Ill.) using objective ×60, and the data were acquired with a Sony digital charge-coupled device camera and processed by Adobe PHOTOSHOP software.

Western blotting was conducted as follows. Two days after transfection, cells were washed with PBS and collected by scraping. They were lysed in ice-cold Tris buffer (50 mM, pH 7.5) containing 5 mM EDTA, 300 mM NaCl, 0.1% Igepal, 0.5 mM NaF, 0.5 mM Na₃VO₄, 0.5 mM PMSF, and antiprotease mixture (Roche Molecular Biochemicals), sonicated, and centrifuged at 13,000 ×g for 10 min. The supernatant was used for protein determination by the Bradford procedure (Bio-Rad) and Western blotting. The proteins were resolved on 12% SDS-polyacrylamide gels, transferred onto nitrocellulose membranes, and incubated with the appropriate antibodies. The anti-GFP antibody was obtained from Santa Cruz Biotechnology and used at a 1/100 dilution. The anti-HA mAb was used at a 1/2,000 dilution. The peroxidase-based detection was performed with Chemiluminescence Reagent (NEN Life Science) according to the manufacturer's instructions.

### Example 5

### Inhibition of Gene Expression with an AAV Vector Encoding an RNA

This Example demonstrates that AAV vectors encoding RNAs of the invention efficiently inhibited expression of genes to which the RNAs were targeted.

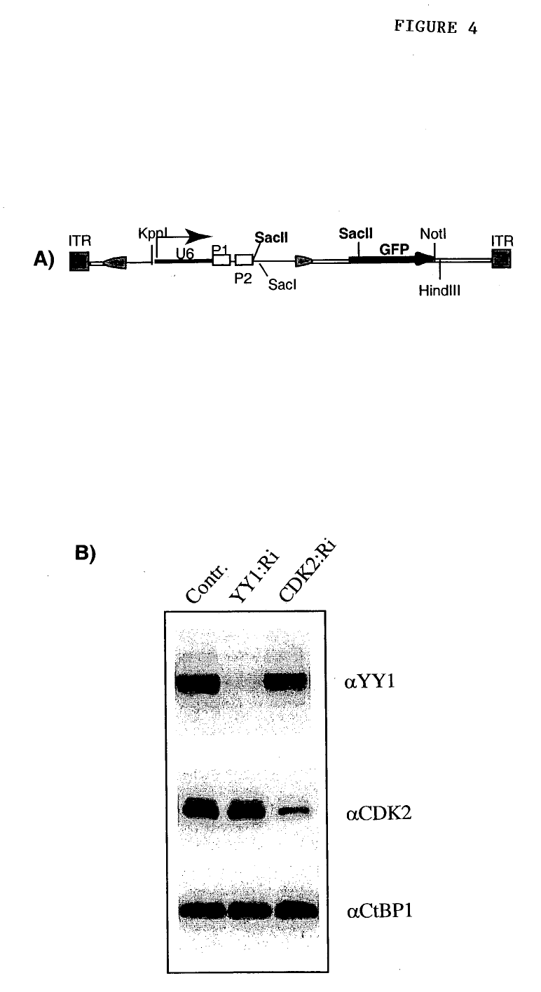

Nucleic acids containing the mouse U6 PolIII promoter (described above) a sequence that is complementary to the YY1 gene (5′ GGGAGCAGAAGCAGGUGCAGA 3′ (SEQ ID NO: 23) or the CDK2 gene (see Examples 1-4), a sequence that is complementary thereto and four thymidines, were inserted into an AAV2 vector containing a sequence encoding GFP (AAV2-GFP vector) (Beck et al. (1999) J. Virol. 73:9446 and Neyns et al. (2001) Intervirology 44:255). The final vector is shown in FIG. 4A. These vectors or the AAV-GFP vector without an insert were used to infect human embryonic kidney 293 cells at a MOI of about 2000. The level of YY1 and CDK2 proteins was then determined using antibodies. As a control for total protein, the amount of CtBP1 was measured. The results, which are set forth in FIG. 4B, show that the YY1 and CDK2 RNAs transcribed from the AAV vector efficiently suppressed the production of YY1 and CDK2 proteins, respectively.

Thus, viral vectors can be used to introduce RNAs that suppress gene expression into cells.

### Example 6

### Selective Silencing of a Dominant Disease Causing Amyotrophic Lateral Sclerosis (ALS) Allele

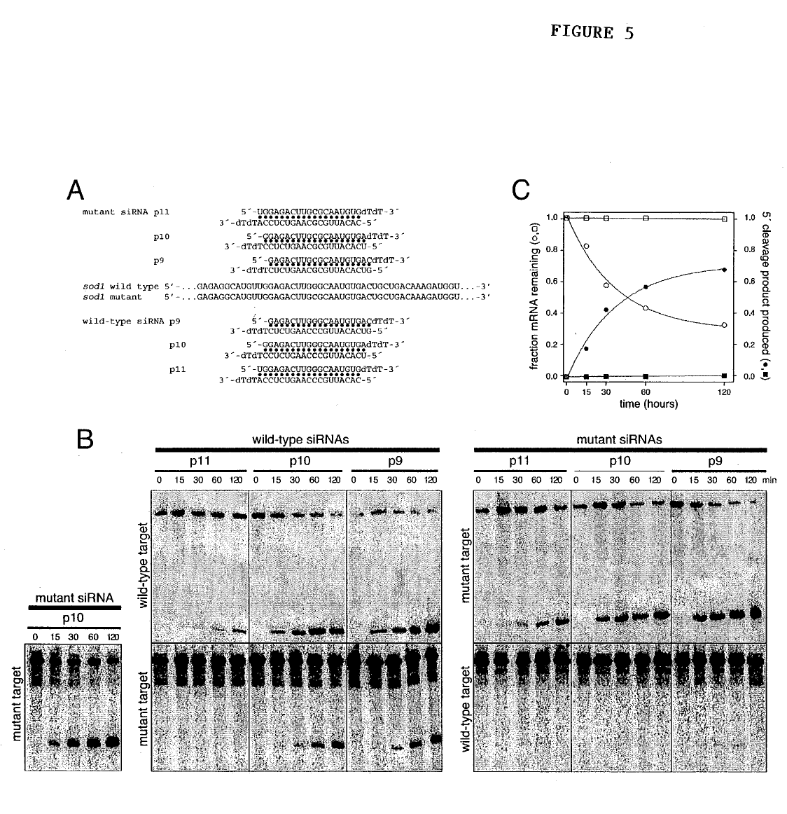

Mechanistic studies suggest that formation of an A-form helix between the siRNA and its mRNA target is required for mRNA cleavage (Chiu, Y.-L. & Rana, T. M. Molecular Cell 10, 549-561 (2002)). We reasoned that mismatches at or near the site of target cleavage would disrupt the required A-form helix. We targeted an allele of SOD1 in which guanosine 256 (G256, relative to the start of translation) is mutated to cytosine, generating a glycine-to-arginine mutation (Gly85Arg). We placed the mismatch at positions 9, 10, and 11 from the 5′ end of the siRNA, and the G256C mutant/wild-type pair produces the largest possible clash (purine:purine) between the mutant siRNA and the wild-type gene and the greatest hydrogen-bonding (G:C) between the mutant siRNA and the mutant SOD1 allele (FIG. 5A). As controls, we synthesized comparable siRNAs to target wild-type but not the mutant SOD1 mRNA (FIG. 5A). In the controls, the siRNAs contain a G:C base pair at the selective site, but the mismatch between wild-type siRNA and mutant allele is a smaller pyrimidine:pyrimidine clash (C:C). The selectivity of each siRNA was tested in a cell-free RNAi reaction containing Drosophila embryo lysate (FIGS. 5B and 5C) (Zamore et al. Cell 101, 25-33 (2000); Tuschl et al. Genes Dev 13, 3191-7 (1999)).

Each of the six siRNAs cleaved the corresponding target RNA, although with dramatically different efficiency (FIG. 5B). For example, both mutant and wild-type p11 siRNAs did not cut their respective RNA targets with a rate expected to be effective in vivo. On the other hand, the p10 mutant siRNA efficiently cleaved the mutant SOD1 mRNA. In all cases, destruction of full-length target mRNA was accompanied by a corresponding accumulation of a ˜288 nt 5′ cleavage product, demonstrating that the siRNAs trigger RNAi, rather than non-specific RNA degradation (FIG. 5B). In the absence of siRNA or in the presence of an unrelated siRNA, the mutant SOD1 target RNA was stable in the Drosophila embryo lysate. Data for both the destruction of target RNA and the accumulation of 5′ cleavage product fit well to a single exponential equation, indicating that the reaction follows pseudo first-order kinetics (FIG. 5C).

To determine the selectivity of the six siRNAs, each siRNA corresponding to the mutant SOD1 sequence was tested for its ability to cleave wild-type SOD1 mRNA, and each wild-type siRNA was tested for its ability to cleave mutant mRNA. Some but not all of the siRNA duplexes effectively discriminated between the target to which they are perfectly matched and the target with which they have a single-nucleotide mismatch (FIG. 5B). We observed two types of defects for a subset of siRNAs. Both wild-type and mutant p11 siRNA did not trigger efficient target cleavage of either the perfectly matched or the mismatched RNA target (FIG. 5B). Thus, these siRNA sequences are inherently poor triggers of RNAi. The p9 and p10 wild-type siRNAs not only triggered rapid cleaveage of their corresponding wild-type target, but also produced significant cleavage of the mutant RNA (FIG. 5B). These siRNAs are good triggers of RNAi but show poor selectivity. In contrast, the p10 mutant siRNA showed both efficient RNAi and robust discrimination between mutant and wild-type SOD1 RNAs, cleaving the mutant far more efficiently than the wild-type RNA in the cell-free reaction (FIGS. 5B and 5C). Because this siRNA showed nearly complete discrimination between mutant and wild-type SOD1 mRNA targets (FIGS. 5B and 5C), it is an ideal candidate for therapeutic application.

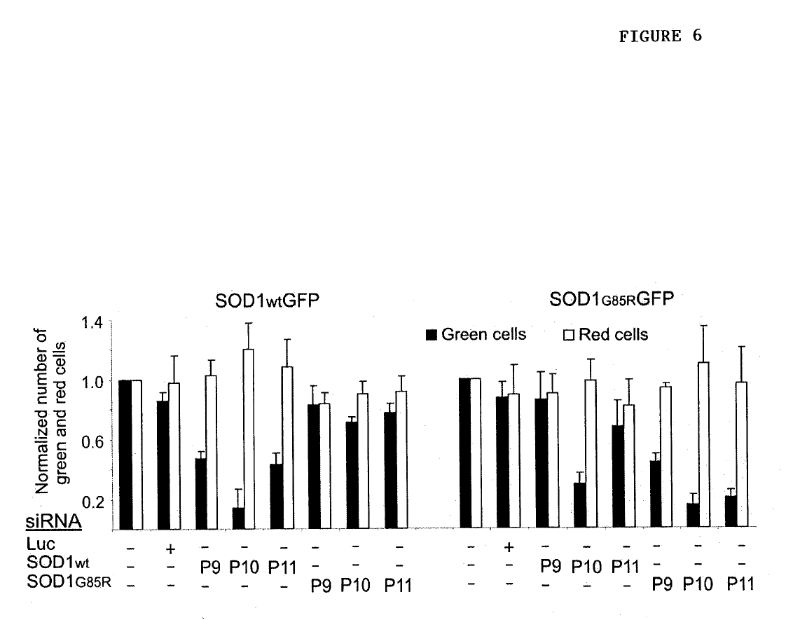

To test whether the cell-free reactions accurately predict siRNA efficacy and selectivity in mammalian cells, we analyzed the siRNAs in a HeLa cell assay. We prepared plasmids expressed either wild-type or G256C (Gly85Arg) mutant SOD1 with GFP fused to the SOD1 carboxyl terminus. Each construct was transfected into Hela cells together with both siRNA and a dsRed-expressing vector that served as both a transfection control and a measure of any non-specific effects of siRNA transfection. The expression of either mutant or wild-type SOD1 was monitored by FACS. Transfection of p9, p10 and p11 siRNAs with their corresponding mutant or wild-type targets suppressed the gene expression, although with distinctly different efficiency and selectivity (FIG. 6). Cotransfection with an siRNA complementary to firefly luciferase did not suppress either SOD1 allele (FIG. 5). As observed in the cell-free reactions, the p10 siRNA against wild-type SOD1 showed no selectivity and suppressed both wild-type and mutant SOD1 mRNA (FIG. 6). The other siRNAs all showed some degree of selectivity, but the p10 siRNA directed against the SOD1 mutant mRNA showed both the greatest efficacy and selectivity, in agreement with the results of the cell-free reactions. Thus, some but not all siRNAs can efficiently discriminate between mRNA targets with a single-nucleotide difference, and these siRNAs can be identified by pre-screening using cell-free RNAi reactions.

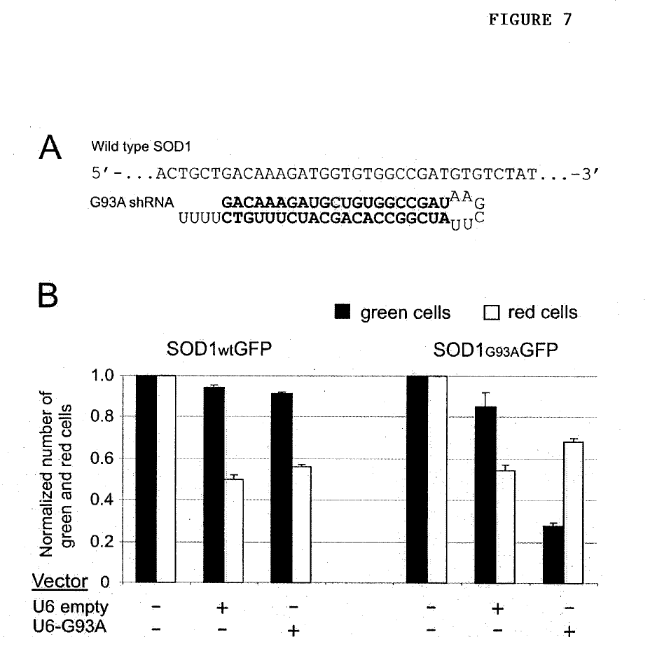

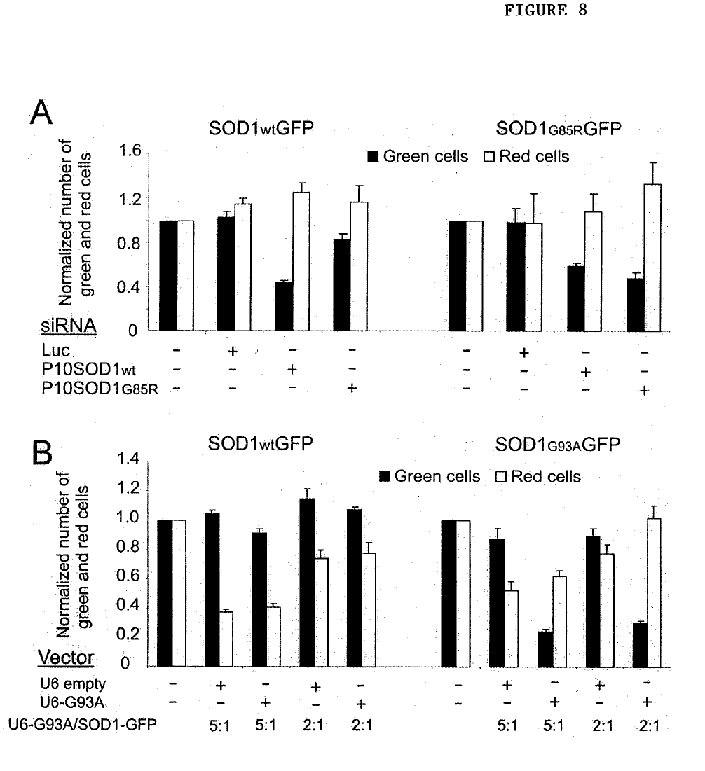

Recently it has been shown that shRNA can trigger RNAi in vivo. To test whether shRNA against mutant SOD1 can selectively block the expression of the mutant but not the wild type SOD1 expression, we constructed a plasmid that synthesizes a shRNA homologous to another disease-causing mutant G281C (Gly93Ala) (Examples 1-4). This mutant was examined because, like the G256C mutation, it places a G:G mismatch at the selective site. We placed the mismatch at position P11, which was in the middle of the siRNA we anticipated that it would produce, and thus are equivalent to P10 in the synthetic siRNA (FIG. 7A). When cotransfected separately with wild-type or mutant SOD1-GFP expressing plasmid, this hairpin construct inhibited mutant expression but not the wild-type (FIG. 7). Thus, hairpin constructs can be used to trigger single-nucleotide selective RNAi of mutant SOD1 in cultured cells. To test if mutant-selective inhibition can be achieved in neuronal cells, we separately transfected the wild-type or mutant SOD1-GFP constructs with either siRNA p10 against mutant G256C or shRNA-synthesizing vector against G281C into the neuroblastoma cell line N2a. As in Hela cells, both synthetic siRNAs and shRNA constructs direct the selective inhibition of mutant SOD1 expression N2a cells (FIGS. 8A, B).

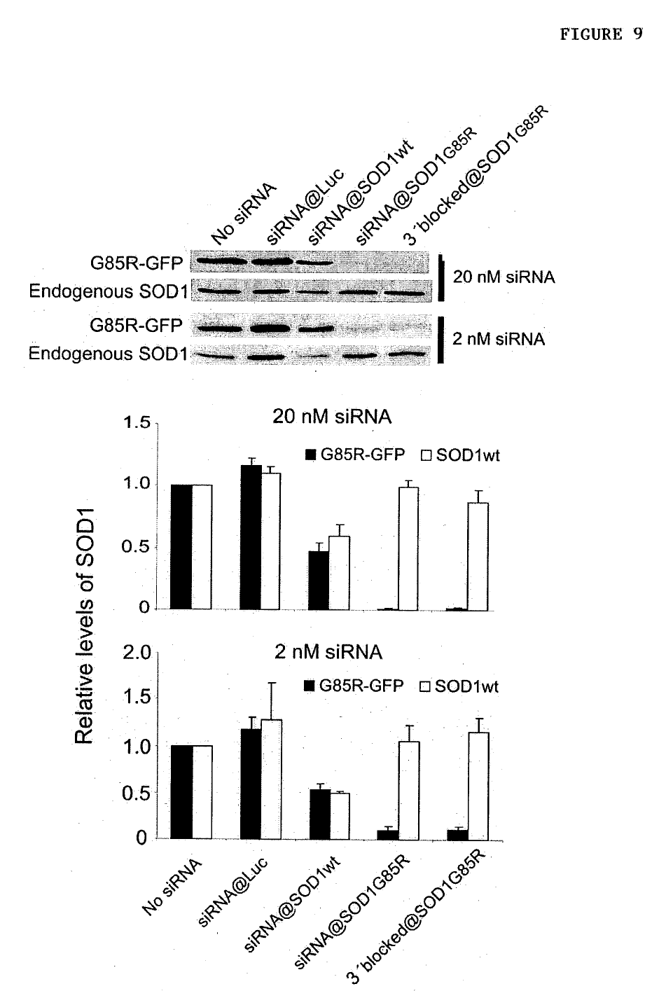

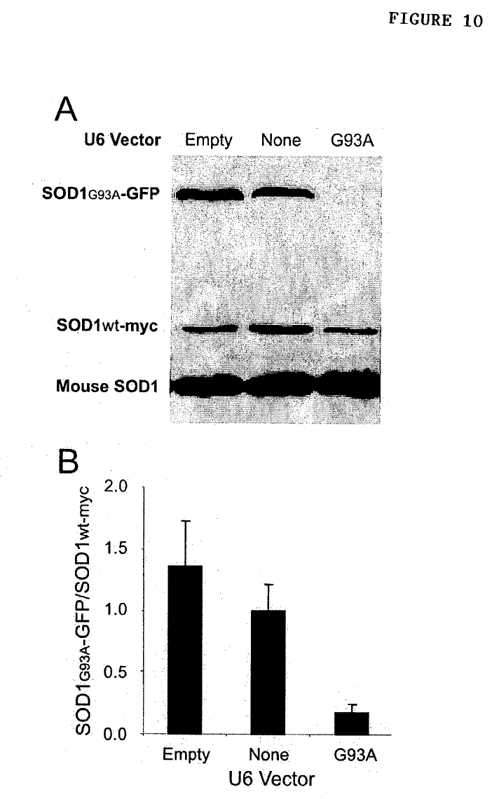

To be therapeutically relevant, single-nucleotide selective siRNAs must discriminate between mutant and wild-type SOD1 when both mRNAs are present in the same cell. We transfected HeLa cells with siRNAs and Gly85Arg mutant SOD1-GFP, and analyzed SOD1 protein expression by immunoblotting with an SOD1-specific antibody that recognizes both the transfected mutant SOD1-GFP fusion protein and endogenous wild-type SOD1. At two different doses, the siRNA inhibited expression of the mutant, but not endogenous wild-type SOD1 (FIG. 9). No additional selectivity was seen with a 3′-blocked siRNA, consistent with reports that siRNAs do not function as primers to trigger the production of ‘secondary siRNAs’ in human cells, as they do in nematodes (Holen, T. et al. Nucleic Acids Res 30, 1757-66 (2002); Chiu, Y.-L. & Rana, T. M. Molecular Cell 10, 549-561 (2002); Schwarz, D. S. et al. Molecular Cell 10, 537-548 (2002); and Sijen, T. et al. Cell 107, 465-76 (2001)).

Finally, to test whether this selective inhibition can occur in vivo, we transfected SOD1 reporters and shRNA plasmid into mice using the hydrodynamic transfection protocol. G281C mutant SOD1-GFP and myc-tagged wild-type human SOD1 expression plasmids were used, enabling detection of mutant and wild-type human SOD1 proteins, as well as the endogenous mouse SOD1 by immunoblotting. We analyzed SOD1 expression in liver, a tissue readily transfectable by the hydrodynamic method. Under these conditions, the shRNA-expresing plasmid selectively decreased mutant but not wild-type human SOD1 expression (FIG. 7).

Thus, this example demonstrates that RNAi can be used to selectively silence a dominant mutant ALS gene. Using multiple siRNAs matching either wild-type or mutant SOD1, we show that siRNAs against mutant SOD1 cleave the mutant, but not the wild-type SOD1 RNA efficiently in vitro. Those siRNAs that show efficacy and selectivity in vitro also selectively inhibited mutant but not wild-type SOD1 protein expression in mammalian cells, even when both the mutant and the wild type proteins are present in the same cells. A vector expressing an RNA that is most likely processed in vivo into an siRNA also selectively inhibited mutant but not wild-type SOD1 expression, even in vivo in mouse liver. These results demonstrate that selective inhibition of a dominant mutant SOD1 allele can be achieved using RNAi.

The results suggest a hypothesis for the molecular basis of siRNA selectivity. The mismatch between the highly selective mutant p10 siRNA and the wild-type SOD1 mRNA creates a G:G clash, whereas the mismatch between the poorly selective wild-type P10 siRNA and the mutant G256C mRNA results in a C:C clash (see FIG. 5A). We predict that a purine:purine mismatch disrupts the A-form helix that is required between the anti-sense strand of the siRNA and its mRNA target (Chiu, Y.-L. & Rana, T. M. Molecular Cell 10, 549-561 (2002)). In contrast, a pyrimidine:pyrimidine mismatch may more readily be accommodated within an A-form helix. Thus, the G:G clash between the siRNA and the wild-type target RNA discriminates against the wild-type target, producing greater selectivity for the mutant target. Furthermore, the presence of a G:C basepair between the mutant siRNA and the mutant target mRNA at the selective site may serve to maximize the energy difference between mismatch and perfect pairing. In support of our hypothesis, an siRNA hairpin vector against a different mutant G281C, also shows good selectivity for mutant SOD1 and creates a G:C basepair with its mutant target and a G:G clash with the wild-type SOD1 mRNA. A more systematic study is now underway to test these ideas, but several published results from other laboratories support this view. In Drosophila embryos, an siRNA having a pyrimidine:purine mismatch (C:A) with its target mRNA was only slightly less effective than the perfectly matched siRNA (Boutla, A. et al. Curr Biol 11, 1776-80 (2001)). Moreover, a separate study showed that an siRNA directed against firefly luciferase failed to produce detectable RNAi in vitro when it contained at position 9 or 10 of its guide strand a purine:purine (A:A) mismatch with its target RNA (Elbashir, S. M. et al. Embo J 20, 6877-88 (2001)). Likewise, an siRNA that showed good selectivity for a mutant Ras mRNA created a purine:purine (A:G) clash with the wild-type allele. Brummelkamp, T. et al. Cancer Cell 2, 243 (2002). Arguing against our hypothesis, one experiment using siRNA against hTF suggests that a G:G mismatch still mediate RNAi (Holen, T., et al. Nucleic Acids Res 30, 1757-66 (2002)). It is possible that this was due to high concentration of siRNA used. Another experiment using shRNA against CDH-1 suggest that a U:C or a U:G mismatch abolished RNAi (Boutla, A., et al. Curr Biol 11, 1776-80 (2001)). In light of our demonstration that small differences in siRNA sequence can produce dramatic differences in efficacy—rather than selectivity—it remains to be shown if these inactive shRNAs were active against a perfectly matched target, and not merely poor triggers of RNAi in general.

Taken together, our data show that both synthetic siRNAs and hairpin vectors can selectively down-regulate the expression of mutant SOD1, even when the mutant mRNA differs from wild-type by only a single nucleotide. We believe that our results show the promise of RNAi as a therapeutic strategy to treat mutant SOD1-induced motor neuron degeneration and ALS. A clear obstacle to such therapeutic application is the delivery of siRNA to the CNS. Because hairpin-expressed siRNA can be readily adapted to viral vectors, it should be possible to develop virus-based delivery systems to treat ALS caused by mutant SOD1 expression (Brummelkamp, T. et al. Cancer Cell 2, 243 (2002); Xia, H. et al. Nat Biotechnol 20, 1006-10 (2002); and Devroe, E. & Silver, P. A. Biotechnol 2, 15 (2002)). In broad terms, our findings are applicable to other CNS diseases that are caused by dominant, gain-of-function type of gene mutations. Finally, our results show that some, but not all siRNAs are capable of single-nucleotide discrimination, and that these siRNAs can be identified by prescreen using cell-free RNAi reactions and cultured cells.

Methods:

RNA and DNA constructs were prepared as follows. Twenty one nucleotide single strand RNAs (FIG. 5) were purchased from Dharmacon Research, deprotected according to manufacturer's instructions, and annealed as described (Nykanen, A. et al. Cell 107, 309-21 (2001)). The 3′-block siRNA was synthesized by addition of a 2′,3′-dideoxy cytocine at the 3′ terminus of the antisense strand. To create wild type and mutant SOD1-GFP fusion proteins, SOD1wt, SOD1_{G85R} and SOD1_{G93A} cDNAs (Dr. Joseph Beckman) were PCR cloned between the PmlI and PstI sites of pCMV/myc/mito/GFP (Invitrogen). This cloning step deleted the mitochondrial targeting sequence. To create myc tagged wild type SOD1, SOD1wt cDNA was PCR cloned between the PstI and XhoI sites of pCMV/myc/mito/GFP. The mitochondrial targeting sequence was then deleted by digestion with BssHII and PmlI and blunt ligation. All constructs were verified by sequencing. DsRed (pDsRed2-C1) was purchased from Clontech (Palo Alto, Calif.). U6-G93A was constructed as described (FIG. 7) (Examples 1-4).

In vitro RNAi assays were conducted as follows: Drosophila embryo lysates were prepared as previously described (Zamore, P. D. et al. Cell 101, 25-33 (2000)). Five hundred and sixty nucleotide human SOD1 target RNAs containing either wild-type or mutant SOD1 G85R coding sequence were cap-labeled using Guanylyl transferase as described previously (Zamore, P. D. et al. Cell 101, 25-33 (2000)). In vitro RNAi reactions were carried out in Drosophila embryo lysate by incubating ˜5 nM of the 5′, P-cap-radiolabeled target RNA with 100 nM duplex siRNA at 25° C. in a standard reaction (Holen, T. et al. Nucleic Acids Res 30, 1757-66 (2002); Zamore, P. D. et al. Cell 101, 25-33 (2000); and Tuschl, T. et al. Genes Dev 13, 3191-7 (1999)). Cleavage products were analyzed on 5% denaturing acrylamide gels, dried, and exposed on image plates (Fuji). Plates were scanned using a Molecular Imager FX (Biorad), and images were analyzed using Quantity One version 4.0.3 (Biorad).

Cell culture and transfections were conducted as follows. Hela cells were cultured in DMEM and N2A cells in DMED and Opti-MEM (1:1), both supplemented with 10% fetal bovine serum (FBS), 100 units ml⁻¹ penicillin, and 100 ug ml⁻¹ streptomycin. Twenty-four hours before transfection, cells (70-90% confluency) detached by trituration, transfered to 6-well plates and cultured in fresh medium without antibiotics. Transfection was carried out using lipofectamine 2000 (invitrogen) according to manufacturer's instructions. The amount of the constructs used in transfections are 4 μg each of mutant or wild type SOD1-GFP and DsRed plasmids, 4×10⁻¹¹ or 4×10⁻¹² mole siRNAs, and 20 or 8 μg U6-G93A.

In vivo transfections were conducted as follows. Twenty four mice 6-8 weeks old were divided into three groups. The first group received no shRNA vector, the second group received 20 μg empty vector and the third group received 20 μg U6-shRNA vector against SOD1 G93A. All groups received both 20 μg of myc tagged human wild type SOD1 and 20 μg GFP tagged SOD1. The vectors were diluted in Ringer's solution so that the total volume equaled 2.5 ml per mouse. Mice were anaesthetized with avertin (240 mg/kg) and the vectors were injected into the tail vein using a 26-gauge needle in less than 10 seconds. Forty eight hours following injection animals were perfused with 5 ml PBS in order to remove blood from the liver. Livers were dissected and quickly frozen on dry ice. Samples were placed in 25 mM PBS buffer (pH 7.2) containing 1% SDS, 1 mM DTT, 1 mM phenylmethylsufonyl fluoride (PMSF), and protease inhibitor cocktail (Sigma, diluted 1:100) and homogenized using a hand held polytrone (Pro-scientific).

Western blot analysis was conducted as follows. Protein concentrations were determined using a BCA protein assay kit (Pierce; Rockville Ill.). Twenty five μg Hela cell proteins or 100 μg liver proteins were separated on a 15% SDS-PAGE gel and transferred onto Genescreen Plus membrane (Perkin Elmer). Rabbit anti-SOD1 (Biodesign) or Sheep anti-SOD1 was the primary and HRP-labeled goat anti-rabbit IgG (Amersham) or donkey anti-sheep IgG was the secondary antibodies. The protein bands were visualized using SuperSignal kit (Pierce) and Kodak Digital Image Station 440CF. The intensity of the bands was quantified using Kodak ID software.

### REFERENCES

1. Fire, A., Xu, S., Montgomery, M. K., Kostas, S. A., Driver, S. E. & Mello, C. C. (1998) Nature (London) 391, 806-811.

2. Bernstein, E., Denli, A. M. & Hannon, G. J. (2001) RNA 7, 1509-1521.

3. Sharp, P. A. (2001) Genes Dev. 15, 485-490.

4. Zamore, P. D. (2001) Nat. Struct. Biol. 8, 746-750.

5. Chuang, C. F. & Meyerowitz, E. M. (2000) Proc. Natl. Acad. Sci. USA 97, 4985-4990.

6. Misquitta, L. & Paterson, B. M. (1999) Proc. Natl. Acad. Sci. USA 96, 1451-1456.

7. Tuschl, T., Zamore, P. D., Lehmann, R., Bartel, D. P. & Sharp, P. A. (1999) Genes Dev. 13,3191-3197.

8. Baglioni, C. & Nilsen, T. W. (1983) Interferon 5, 23-42.

9. Williams, B. R. (1997) Biochem. Soc. Trans. 25, 509-513.

10. Paddison, P. J., Caudy, A. A. & Hannon, G. J. (2002) Proc. Natl. Acad. Sci. USA 99, 1443-1448.

11. Billy, E., Brondani, V., Zhang, H., Muller, U. & Filipowicz, W. (2001) Proc. Natl. Acad. Sci. USA 98, 14428-14433.

12. Elbashir, S. M., Harborth, J., Lendeckel, W., Yalcin, A., Weber, K. & Tuschl, T. (2001) Nature (London) 411, 494-498.

13. Bogenhagen, D. F., Sakonju, S. & Brown, D. D. (1980) Cell 19,27-35.

14. Elbashir, S. M., Lendeckel, W. & Tuschl, T. (2001) Genes Dev. 15, 188-200.

15. Tavemarakis, N., Wang, S. L., Dorovkov, M., Ryazanov, A. & Driscoll, M. (2000) Nat. Genet. 24, 180-183.

16. Kennerdell, J. R. & Carthew, R. W. (2000) Nat. Biotechnol. 18, 896-898.

17. Bernstein, E., Caudy, A. A., Hammond, S. M. & Hannon, G. J. (2001) Nature (London) 409, 363-366.

18. Ketting, R. F., Fischer, S. E., Bernstein, E., Sijen, T., Hannon, G. J. & Plasterk, R. H. (2001) Genes Dev. 15, 2654-2659.

19. Knight, S. W. & Bass, B. L. (2001) Science 293, 2269-2271.

20. Kunkel, G. R., Maser, R., Calvet, J. & Pederson, T. (1986) Proc. Natl. Acad. Sci. USA 83, 8575-8579.

21. Harper, J. W. & Adams, P. D. (2001) Chem. Rev. 101, 2511-2562.

22. Li, E., Bestor, T. H. & Jaenisch, R. (1992) Cell 69, 915-926.

Equivalents

It will be apparent to those skilled in the art that the examples and embodiments described herein are by way of illustration and not of limitation, and that other examples may be used without departing from the spirit and scope of the present invention, as set forth in the claims.

1. A nucleic acid comprising the following nucleotide sequences in a 5′ to 3′ order: an RNA polymerase promoter sequence; a first target sequence that is essentially complementary to a sequence of a target nucleic acid or complement thereof; a spacer sequence; a second target sequence that is essentially complementary to the first target sequence; and an RNA polymerase termination signal, wherein an RNA transcribed from the nucleic acid can inhibit expression of the target gene.

2. The nucleic acid of claim 1, wherein the RNA transcribed from the nucleic acid forms a hairpin structure.

3. The nucleic acid of claim 1, wherein the polymerase is RNA polymerase III (Pol III) and the polymerase termination signal comprises a number of thymidines sufficient for arresting Pol III activity.

4. The nucleic acid of claim 1, wherein the first target sequence is at least about 95% identical to a nucleotide sequence of the target nucleic acid or the complement thereof.

5. The nucleic acid of claim 4, wherein the first target sequence is perfectly complementary to a sequence of a target nucleic acid or the complement thereof.

6. The nucleic acid of claim 4, wherein the target nucleic acid is a target gene.

7. The nucleic acid of claim 1, wherein the first and the second target sequences comprise from about 15 to about 30 nucleotides.

8. The nucleic acid of claim 7, wherein the first and the second target sequences comprise from about 19 to 25 nucleotides.

9. The nucleic acid of claim 1, wherein the first target sequence comprises a portion of the coding sequence of the target nucleic acid or the complement thereof.

10. The nucleic acid of claim 1, wherein the first and the second target sequences differ in at most 2 nucleotides.

11. The nucleic acid of claim 1, wherein the first and the second target sequences are perfectly complementary.

12. The nucleic acid of claim 3, wherein the number of thymidines sufficient for arresting Pol III activity is 4 or 5 thymidines.

13. The nucleic acid of claim 1, wherein the spacer sequence consists of about 3 to about 15 nucleotides.

14. The nucleic acid of claim 13, wherein the spacer sequence consists of about 5 to about 10 nucleotides.

15. The nucleic acid of claim 14, wherein the spacer consists of about 6 nucleotides.

16. The nucleic acid of claim 1, wherein the spacer sequence comprises a palindromic sequence.

17. The nucleic acid of claim 16, wherein the palindromic sequence is AACGTT.

18. The nucleic acid of claim 3, wherein the Pol III promoter comprises a U6 promoter.

19. The nucleic acid of claim 18, wherein the Pol III promoter comprises from about nucleotide −315 to about nucleotide +1 of the mouse U6 promoter (SEQ ID NO: 3).

20. The nucleic acid of claim 1, which is DNA.

21. The nucleic acid of claim 1, which is in a plasmid.

22. The nucleic acid of claim 1, which is in an expression vector.

23. The nucleic acid of claim 22, wherein the expression vector is a eukaryotic expression vector.

24. The nucleic acid of claim 23, wherein the eukaryotic expression vector is a mammalian expression vector.

25. The nucleic acid of claim 24, wherein the eukaryotic expression vector is a viral vector.

26. The nucleic acid of claim 25, wherein the viral vector is an adenoviral vector.

27. The nucleic acid of claim 1, wherein the polymerase is a Pol III; the first target sequence is essentially complementary to a sequence of a target nucleic acid or complement thereof; the first and the second target sequences consist of about 19-23 nucleotides and are perfectly complementary to each other; the spacer sequence consists of about 6 nucleotides; and the RNA polymerase termination signal consists of 4 or 5 thymidines.

28. A nucleic acid comprising the following nucleotide sequences in a 5′ to 3′ order: a Pol III promoter sequence; a first restriction enzyme recognition sequence; a spacer sequence; a second restriction enzyme recognition sequence; and a number of thymidines sufficient for arresting Pol III activity, wherein an RNA molecule transcribed from the nucleic acid in which a first and a second target sequences are inserted in the first and second restriction enzyme recognition site, respectively, inhibits expression of a target gene comprising a sequence that is essentially complementary to the first or the second target sequence.

29. The nucleic acid of claim 28, wherein the spacer sequence consists of about 3 to about 15 nucleotides.

30. The nucleic acid of claim 29, wherein the spacer sequence consists of about 5 to about 10 nucleotides.

31. The nucleic acid of claim 30, wherein the spacer sequence consists of about 6 nucleotides.

32. The nucleic acid of claim 28, wherein the spacer sequence comprises a palindromic sequence.

33. The nucleic acid of claim 32, wherein the palindromic sequence is AACGTT.

34. The nucleic acid of claim 28, wherein the Pol III promoter comprises a U6 promoter.

35. The nucleic acid of claim 34, wherein the U6 promoter comprises from about nucleotide −315 to about nucleotide +1 of the mouse U6 promoter (SEQ ID NO: 3).

36. The nucleic acid of claim 28, further comprising at least one additional restriction enzyme recognition sequence between the Pol III promoter and the first restriction enzyme recognition sequence.

37. The nucleic acid of claim 28, further comprising at least one additional restriction enzyme recognition sequence between the second restriction enzyme recognition sequence and the thymidines sufficient for arresting Pol III activity.

38. An RNA comprising the following nucleotide sequences in a 5′ to 3′ order: a first target sequence of about 19 to about 25 nucleotides, which is at least about 95% identical to a portion of a nucleotide sequence of a target nucleic acid or the complement thereof; a spacer sequence of about 5 to 10 nucleotides; a second target sequence of about 19 to about 25 nucleotides that is essentially complementary to the first target sequence; and at least a portion of an RNA polymerase termination signal, wherein the RNA inhibits expression of a target gene comprising a sequence that is essentially complementary to the first or the second target sequence.

39. The RNA of claim 38, wherein the RNA forms a hairpin structure.

40. The RNA of claim 38, wherein the first and the second target sequences consist of about 19 to about 23 nucleotides and are perfectly complementary to each other; the first target sequence is perfectly complementary to a sequence of the target nucleic acid or complement thereof; and the polymerase termination signal consists of 4 or 5 uridines.

41. A cell comprising the nucleic acid of claim 1.

42. A cell comprising the nucleic acid of claim 27.

43. The cell of claim 41, which is a eukaryotic cell.

44. The cell of claim 42, which is a eukaryotic cell.

45. The cell of claim 43, which is a mammalian cell.

46. The cell of claim 44, which is a mammalian cell.

47. The cell of claim 41, which is an isolated cell.

48. The cell of claim 42, which is an isolated cell.

49. A method for preparing a nucleic acid for inhibiting the synthesis of a target protein in a eukaryotic cell, comprising (i) providing a nucleic acid of claim 25; and (ii) introducing into the first restriction recognition sequence a first oligonucleotide of about 15-30 nucleotides comprising a sequence that is essentially complementary to a sequence of the target nucleic acid.

50. The method of claim 49, further comprising introducing into the second restriction recognition sequence a second oligonucleotide of about 15-30 nucleotides that is essentially complementary to the sequence of the first oligonucleotide.

51. The method of claim 49, wherein the first oligonucleotide comprises about 20 to 23 consecutive nucleotides of the target nucleic acid or the complement thereof.

52. The method of claim 51, further comprising introducing into the second restriction recognition sequence a second oligonucleotide comprising a nucleotide sequence that is perfectly complimentary to the sequence of the first oligonucleotide.

53. A method for producing RNA molecules that inhibit expression of a target nucleic acid in a eukaryotic cell, comprising introducing into a eukaryotic cell a nucleic acid of claim 1, wherein the first target sequence is essentially complementary to a sequence of the target nucleic acid or the complement thereof, such that the nucleic acid is transcribed in the eukaryotic cell and produces RNA molecules that inhibit expression of a target nucleic acid.

54. The method of claim 53, wherein the first target sequence is perfectly complementary to a sequence of the target nucleic acid and the first and the second target sequences consist of about 19 to 25 nucleotides and are perfectly complementary to each other.

55. A method for inhibiting the synthesis of a target protein in a eukaryotic cell, comprising introducing into a target cell a nucleic acid of claim 1, wherein the first target sequence is essentially complementary to a sequence of the nucleic acid encoding the target protein or the complement thereof, such that the nucleic acid is transcribed in the target cell and thereby inhibits the synthesis of the target protein.

56. A method for inhibiting the synthesis of a target protein in a eukaryotic cell, comprising introducing into a target cell a nucleic acid of claim 27, wherein the first target sequence is perfectly complementary to a sequence of the nucleic acid encoding the target protein or the complement thereof, such that the nucleic acid is transcribed in the target cell and thereby inhibits the synthesis of the target protein.

57. The method of claim 55, wherein the cell is an isolated cell.

58. The method of claim 55, wherein the cell is an isolated cell.

59. A method for inhibiting the synthesis of a target protein in a cell of a subject, comprising introducing into the cell of the subject a nucleic acid of claim 1, wherein the first target sequence is essentially complementary to a sequence of the gene encoding the target protein or the complement thereof, such that the nucleic acid is transcribed in the target cell and thereby inhibits the synthesis of the target protein.

60. A method for inhibiting the synthesis of a target protein in a cell of a subject, comprising introducing into the cell of the subject a nucleic acid of claim 27, wherein the first target sequence is perfectly complementary to a sequence of the gene encoding the target protein or the complement thereof, such that the nucleic acid is transcribed in the target cell and thereby inhibits the synthesis of the target protein.

61. The method of claim 57, comprising first obtaining the cell from a subject; introducing the nucleic acid into the cell ex vivo and administering the cell to the subject.

62. The method of claim 60, comprising first obtaining the cell from a subject; introducing the nucleic acid into the cell ex vivo and administering the cell to the subject.

63. A kit for inhibiting the synthesis of a target protein in a cell, comprising a nucleic acid of claim 1 and at least one reagent for introducing the nucleic acid into a cell.

64. A kit for inhibiting the synthesis of a target protein in a cell, comprising a nucleic acid of claim 28 and at least one reagent for introducing the nucleic acid into a cell.
